(19) United States
(12) Patent Application Publication
(10) Pub. No.: US 2013/0216553 A1
(43) Pub. Date: Aug. 22, 2013

(54) Compositions and Methods for Modulation and Detection of Immune and Inflammatory Responses

(71) Applicants: Robert B. Clark, Farmington, CT (US); Frank C. Nichols, West Hartford, CT (US)

(72) Inventors: Robert B. Clark, Farmington, CT (US); Frank C. Nichols, West Hartford, CT (US)

(73) Assignee: University of Connecticut, Farmington, CT (US)

(21) Appl. No.: 13/882,239

(22) PCT Filed: Oct. 31, 2011
(86) PCT No.: PCT/US2011/058551

§ 371 Date: May 3, 2013

Related U.S. Application Data
(60) Provisional application No. 61/455,967, filed on Oct. 29, 2010.

(51) Int. Cl. G01N 33/92; A61K 39/02; A61K 39/40

(52) U.S. Cl. CPC G01N 33/92; A61K 39/40; A61K 39/02
USPC 424/164.1; 435/29; 530/389.5; 564/15; 424/234.1

(57) ABSTRACT

A method for detecting an inflammatory or an autoimmune condition, comprising analyzing bacterial lipids, such as phosphorylated dihydroceramides (PDHC), in a sample; and, comparing results of the analysis of the bacterial lipids in the sample with information on occurrence of the bacterial lipids in a comparable sample, wherein the comparison is indicative of the inflammatory or the autoimmune condition. An example of the autoimmune condition is multiple sclerosis. According to one embodiment, an increased ratio of phosphoglycerol dihydroceramide (PG DHC) to phosphoethanolamine dihydroceramide (PE DHC) in a blood sample indicates a presence of MS in the source patient. The use of PDHCs as biomarkers for detection of MS is described. Antibodies specific to PG DHC or PE DHC are also provided, along with their uses. Also provided are compositions comprising bacteria-originated lipids useful for modulation of immune responses or TLR pathways in humans, animals, and human or animal cells or tissues.

### FIELD

This application relates to the general field of compositions and methods for modulation and detection of immune and inflammatory responses.

### BACKGROUND

A number of recent reports focused on the role of bacteria commonly inhabiting human bodies, or commensal bacteria, in the functioning of immune system and human disease. In particular, commensal bacterial were implicated in development and regulation of inflammatory and autoimmune diseases or conditions. See, for example, Wen et al. “Innate immunity and intestinal microbiota in the development of Type 1 diabetes” Nature 455:1109-1113 (2008); Yokote et al. “NKT cell-dependent amelioration of a mouse model of multiple sclerosis by altering gut flora” Am. J. Pathol. 173:1714-1723; Mazmanian et al. “A microbial symbiosis factor prevents intestinal inflammatory disease” Nature 453:620-625 (2008). However, this information was not translated into useful medical or diagnostic applications.

Inflammatory responses characterize a large group of normal and pathologic diseases and conditions in humans or animals. Inflammatory responses are a group of complex biological responses, which typically involve vascular changes, of animal cells and tissues to harmful stimuli, such as pathogens, damaged cells, or irritants. Immune system involvement in some inflammatory responses, such as those seen in allergies and autoimmune disorders, is well known. Involvement of the immune system in some other inflammatory events, such us those observed in cancer, atherosclerosis, and ischemic heart disease, is less well established, although such a possibility is recognized. Inflammatory events involve a large variety of tissue, cellular and molecular events and mechanisms. A number of useful inflammation biomarkers are known, but there is a continuing need for both clinical and research biomarkers, and methods for assessing inflammatory states that would possess improved reproducibility, biological variability, analytic variability, sensitivity and specificity, as well as large-scale feasibility.

Autoimmune diseases and conditions are a large group of diseases and conditions, which includes dozens of important and debilitating human diseases and disorders in which the immune system attacks the host's own tissues and cells. It is thought that each autoimmune disease is most likely caused by a combination of different factors, and even classification of a disease or a condition as autoimmune is complicated. For example, according to one convention accepted in the medical field, for a disease to be regarded as an autoimmune disease, it needs to answer to the so-called Witebsky's postulates, first formulated by Ernst Witebsky and colleagues in 1957, which include direct evidence from transfer of pathogenic antibody or pathogenic T cells, indirect evidence based on reproduction of the autoimmune disease in experimental animals, and circumstantial evidence from clinical clues. Examples of diseases typically regarded as autoimmune are rheumatoid arthritis, systemic lupus erythematosus (SLE), diabetes (type 1), and multiple sclerosis. Autoimmune diseases often have variable symptoms and courses and do not always restrict themselves to one part of the body. For example, SLE can affect the skin, joints, kidneys, heart, nerves, blood vessels, and more. In some patients, rheumatoid arthritis can affect the heart, blood vessels and lungs, in addition to the joint problems it typically causes. Autoimmunity may also play a role in the development of atherosclerosis. While it is currently understood that the immune system in most individuals has the potential to attack self-tissues, the factors that lead to autoimmune diseases in only a subset of individuals remain unknown. The difficulties in classifying and diagnosing autoimmune diseases and condition contribute to a continuing need for biomarkers and methods for diagnosing and assessing autoimmune diseases and conditions, both in the clinical and research contexts.

For some relatively common autoimmune diseases, no biomarkers are currently known and no straightforward diagnostic methods exist. One such disease is multiple sclerosis (MS), which is generally considered to be an autoimmune disease. MS is currently characterized as a human disease in which the immune system targets and attacks the myelin sheath that surrounds and protects the nerve fibers of the central nervous system (CNS). The resulting damage to the myelin and the nerve fiber greatly disrupts the normal flow of electrical impulses to and from the brain, resulting in the various symptoms of MS.

The diagnosis of MS is very difficult and there is no single test that confirms MS in a patient. Typically, physicians require a detailed medical history including the symptoms experienced by the patient; a careful physical exam, including tests of coordination, strength and reflexes; and a number of laboratory tests on samples of blood or cerebrospinal fluid (CSF) to try to rule out other possible causes for the symptoms experienced by the patient. A preferred test is magnetic resonance imaging (MRI) of the brain, which can detect plaques, lesions or scarring which might be caused by MS. However, MRIs have problems with both sensitivity and specificity. A test of Visual Evoked Potentials (VEP), which studies the speed of electrical signals in parts of the brain, may also be used. However, a course and progression of MS is highly variable between patients and is very hard to predict for a given individual. Many patients experience episodes of serious disease symptoms separated by months or more of at least partial remission. At present, there are no known biological markers or methods employing such biomarkers that predict disease activity for MS.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

Disclosed herein are methods for detecting a disease or a condition, or detection methods, which involve, in any combination, detecting, testing or analyzing bacterial lipids present in a cell or a tissue sample obtained from a human or an animal. In some embodiments of the present invention, bacterial lipids under analysis are bacterial lipids that are not synthesized by the human or the animal, which are referred to as “bacteria-originated lipids.” In one variation, bacteria-originated lipids are synthesized by commensal bacteria living in various parts of human or animal organisms.

Detection of a disease or a condition according to various embodiments of the detection methods disclosed herein can employ appropriate analytical methods, techniques or procedures. In some embodiments of the detection methods, mass-spectrometry is employed in the analysis of bacterial lipids. In some other embodiments, immunochemical techniques are employed.

According to some of the embodiments of the present invention, bacteria-originated lipids are used as biological markers, or biomarkers, for detection of diseases and conditions. For example, patterns of bacteria-originated lipids detected by an analytical method in a sample obtained from a human or animal correlate with a presence, absence, state or degree of a disease or condition. Such patterns therefore can be used in the methods for detecting diseases and conditions.

Also disclosed herein are antibodies against bacteria-originated lipids and uses of such antibodies. For example, antibodies against bacteria-originated lipids are used in methods of detecting a disease or a condition, methods of modulating immune or inflammatory responses in the humans or the animals or in the human or animal cells, or in therapeutic and diagnostic methods related to diseases and conditions. Antibodies against the bacteria-originated lipids are also used in medicaments, pharmaceutical compositions, research, analytical and diagnostic compositions, tools, kits and reagents related to treatment and detection of various diseases and conditions or modulation of immune or inflammatory responses in human or animal cells and organisms, as well and in the research activities related to such treatment and detection.

Some embodiments of the methods described herein are methods for detection of inflammatory or autoimmune diseases, conditions or states. Examples of such inflammatory diseases, conditions or states are provided elsewhere in this document. Some other embodiments of the methods disclosed herein are useful for detection of multiple sclerosis, or MS. One such embodiment is a method for detecting MS biomarkers. In one example, the method for detecting MS biomarkers employs an analysis of a blood sample. The method is useful for diagnosing, assessing, monitoring, following the progression of MS. It is also useful in MS prognosis and prediction. For example, it is useful for predicting and exacerbation of symptoms in patients with MS. The method is also useful for monitoring and evaluating the efficacy of clinical treatments for MS. Generally, the methods, biomarkers, molecules, such as antibodies, and other elements disclosed herein provide the first blood test for detection of MS.

As disclosed herein, patients with MS have a pattern of bacteria-originated lipids in samples of some of their tissues, such as blood and brain tissues, or in bacterial samples obtained from the patients' bodies, the pattern being detectably different from a pattern of bacteria-originated lipids in the corresponding samples obtained from MS-free control subjects. By way of example, some of the bacteria-originated lipids originate from commensal bacteria, such as Porphyromonas gingivalis that is often present in the oral cavity. Among the novel lipids of such bacteria are phosphorylated dihydroceramides (PDHCs). Two major classes of PDHCs are phosphoethanolamine dihydroceramides (PE DHCs) and phosphoglycerol dihydroceramides (PG DHCs). These two lipid classes have different biological activities related to specific structural components present in each class.

In one exemplary embodiment of the present invention, the bacterial lipids present in human serum or other fluids are characterized and quantitated using MRM (multiple reaction monitoring) mass spectrometry. MRM-mass spectrometry is the approach used in this embodiment because it provides the advantages of most specific identification and quantification of the lipid families. The methods disclosed herein include analysis of samples of obtainable bodily fluids, specifically serum and cerebrospinal fluid, but also including synovial fluid, tears, and lymphatic fluid. Tissue samples may also be assessed by the disclosed methods. In an exemplary embodiment, monoclonal antibodies are generated to specific PDHC lipids, and such monoclonal antibodies are used in an ELISA to detect the presence, quantity and pattern of serum bacterial lipids in an individual.

Also disclosed herein are compositions comprising bacteria-originated lipids useful for modulation of immune or inflammatory responses, activation of toll-like receptors (TLRs) or modulation of their activity, as well as modulation of toll-like receptor signaling pathways (“TLR pathways”) and binding to TLRs in humans, animals, and human or animal cells tissues, along with corresponding methods and uses of such compositions. According to some embodiments of the present invention, bacteria-originated lipids are used in medicaments, pharmaceutical compositions, research, analytical and diagnostic compositions, tools, kits and reagents related to treatment and detection of various diseases and conditions, modulation of immune or inflammatory responses, modulation of TLR pathways, binding to TLRs, and in the therapeutic, diagnostic and research activities related to immune and inflammatory pathways, TLRs and TLR pathways, and any related diseases, conditions or states.

### BRIEF DESCRIPTION OF THE DRAWINGS

Illustrative embodiments of the present invention are described in detail below with reference to the following drawing figures:

FIG. 1 is a schematic representation of the chemical structures of bacterial PDHCs.

FIG. 2 is a bar graph schematically representing the results of the analysis of bacteria-originated PDHCs recovered from intestinal and oral bacterial samples. The ion abundances of high and low mass PDHC lipid classes were summed and the recovery of each lipid class is depicted as the percent of the total ion abundance of the quantified PDHCs. Standard deviation bars are shown for lipid extracts from Bacteroides vulgatus (n=13), Prevotella capri (n=2), and Porphyromonas gingivalis (n=6).

FIG. 3 is a bar graph schematic representation of the analysis of bacteria-originated PDHCs recovered from subgingival plaque samplds (n=2), samples of healthy/mildly inflamed gingival tissue (GT H+G, n=7), periodontitis gingival tissue samples (GT Perio, n=6), blood plasma samples from periodontally healthy subjects (Blood Cont, n=8), blood plasma from patients with generalized severe periodontitis (Blood Perio, n=7), carotid atheroma (Atheroma, n=11) and postmortem brain samples from non-MS subjects (Brain Control, n=14). The ion abundances of high and low mass PDHC lipid classes were summed and the recovery of each lipid class is depicted as the percent of the total ion abundance of the quantified PDHC lipids. Standard deviation bars are shown.

FIG. 4 is a bar graph schematically representing the results of the analysis of bacteria-originated PCHCs in paired patent artery and atheroma samples. For each carotid atheroma, the patent artery segment of the proximal common carotid artery was excised from the gross atheroma located within the carotid sinus. A defined amount (approximately 3 μg of total lipids in 5 μl of HPLC solvent) of each lipid extract was analyzed by MRM MS/MS and the recovery of each lipid class is depicted as the percent of the total ion abundance of the quantified PDHC lipids. The mean PDHC abundances and the standard error are depicted for five paired control and atheroma lipid extracts.

FIG. 5 is a dot plot schematically representing the results of PDHC lipid analysis of brain samples obtained from active MS patients and control patients. Frozen brain samples from control patients (n=13) and MS patients with active disease (n=12) were analyzed for the presence bacteria-originated PE DHC and PG DHC using MRM-MS. The PG DHC/PE DHC total ion abundance ratios were calculated using the summed ion recoveries from pooled HPLC fractions.

FIG. 6 is a dot plot schematically representing the results of PDHC lipid analysis of serum samples obtained from active MS patients and control patients. Serum samples from control (n=16) and MS patients (n=19) were analyzed for the presence of bacteria-originated PE DHC and PG DHC using MRM-MS. PG DHC/PE DHC total ion abundance ratios were calculated using the ion abundances recovered from samples of the total serum lipid extracts.

FIG. 7 is a line plot illustrating enhancement of experimental allergic encephalomyelities (EAE) by P. gingivalis total lipid (TL) and the PE DHC lipid fraction in female C57BL/6 wild-type (WT) mice aged 4-8 weeks, which were immunized subcutaneously with MOG35-55 peptide (100-200 μg/mouse) in CFA containing 500 μg of H37Ra mycobacteria on day 0. Mice also received Ptx intravenously (150-250 ng) on days 0 and 2. On day 0, mice also received a single 20-μl intraperitoneal (i.p.) injection of EtOH, P. gingivalis TL (2.5 μg), or P. gingivalis PE DHC (250 ng). EAE was graded as follows: grade 1, tail paralysis; grade 2, abnormal gait; grade 3, hind limb paralysis; grade 4, hind and front limb paralysis; grade 5, death. The results illustrated are from one representative experiment each and are depicted as the average EAE score of a given cohort of mice on each day after immunization.

FIG. 8 is a line plot illustrating enhancement of EAE by P. gingivalis total lipid (TL) and the PE DHC lipid fraction in female WT and IL-15−/− mice aged 4-8 weeks, which were immunized subcutaneously with MOG35-55 peptide (100-200 μg/mouse) in CFA containing 500 μg of H37Ra mycobacteria on day 0. Mice also received Ptx intravenously (150-250 ng) on days 0 and 2. On day 0, mice also received a single 20-μl i.p. injection of EtOH, P. gingivalis TL (2.5 μg), or P. gingivalis PE DHC (250 ng). Additional WT mice also received a single 20-μl i.p. injection of the control lipid, bovine sphingomyelin (250 ng). EAE was graded as discussed above. The results illustrated are from one representative experiment each and are depicted as the average EAE score of a given cohort of mice on each day after immunization.

FIG. 9 is a line plot illustrating enhancement of EAE by P. gingivalis total lipid (TL) and the PE DHC lipid fraction in WT and IL-15Rα−/− in female mice aged 4-8 weeks, which were immunized subcutaneously with MOG35-55 peptide (100-200 μg/mouse) in CFA containing 500 μg of H37Ra mycobacteria on day 0. Mice also received Ptx intravenously (150-250 ng) on days 0 and 2. On day 0, mice also received a single 20-μl i.p. injection of EtOH, P. gingivalis TL (2.5 μg), or P. gingivalis PE DHC (250 ng). EAE was graded as discussed above. Results illustrated are from one representative experiment each and are depicted as the average EAE score of a given cohort of mice on each day after immunization.

FIG. 10 is a line plot The PE DHC lipid fraction fails to enhance EAE in TLR2−/− mice. EAE was induced and graded as discussed above using wild-type (WT) or TLR2−/− mice. On day 0, wild-type and TLR2−/− mice received a single 20-μl i.p. injection of EtOH or P. gingivalis PE DHC (250 ng). Results illustrated are a composite of studies (WT mice, n=28; TLR2−/− mice, n=15) and represent the average EAE score for each group (±SEM for wild-type mice) on each day after immunization.

FIG. 11 is a plot schematically illustrating the results of electrospray MS analysis of PE DHC lipids recovered from P. gingivalis. Total lipids of P. gingivalis were isolated and fractionated by high performance liquid chromatography (HPLC). Fractions containing the characteristic molecular ions of PE DHC lipids were pooled and repurified by HPLC. Repurified fractions demonstrating 705, 699, and 677 negative ions were pooled. The structure of the high-mass PE DHC lipid (705 m/z) is shown in the inset with the component fatty acid and long-chain base structures identified. The lower-mass PE DHC lipids indicated by 691 or 677 m/z ions contain 18 carbon or 17 carbon long-chain bases, respectively, as previously described. 4. The plot shows the absence of ions characteristic for lipid A moieties produced by P. gingivalis (1195, 1435, 1449, 1690, and 1770 m/z negative ions).

FIG. 12 is a dot plot, which illustrates the results of the animal study demonstrating that administration of PE DHC resulted in increased recovery of bacterial lipids in the brains of mice with EAE. PBS, EtOH, or PE DHC-injected mice (25 ng, 250 ng, or 2.5 μg) were sacrificed after day 20 post-EAE immunization. The brains of these mice were removed, extracted for phospholipids, and 3-OH isoC_{17:0} fatty acid quantified using negative ion chemical ionization gas chromatography-mass spectrometry. The average 3-OH isoC_{17:0} recovery (three determinations per mouse brain sample) as a function of both the treatment and final EAE score was depicted as picograms of 3-OH isoCl_{7:0} per 0.5 mg of total brain lipid extracted. The average SEM for all brain lipid determinations was ±2.2 pg/0.5 mg total lipid.

FIG. 13 is a bar graph, which illustrates the results of an in vitro study demonstrating that the PE DHC lipid fraction activated APCs and induced IL-6 secretion in vitro in a TLR2-dependent manner. Bone marrow-derived DCs from wild-type (WT) or TLR2−/− mice were cultured alone or with plate-bound EtOH, LPS (1 μg), MMP (10 μg), or PE DHC (2.5 μg). After 18 hours, culture supernatants were assayed for IL-6 via enzyme-linked immunosorbent assay. Histogram bars depict the mean±SD (n=4 trials).

FIG. 14 is a two dimensional dot plot illustrating the data obtained from a flow cytometry analysis which illustrates the results of an in vitro study demonstrating that the PE DHC lipid fraction activated APCs and induced IL-6 secretion in vitro in a TLR2-dependent manner. Naïve CD4+CD25− wild-type Teff (0.25×106/well) were cultured with irradiated wild-type or TLR2−/− Tds as a source of antigen presenting cells (0.75×106/well), anti-CD3 antibody (1 μg/ml), granulocyte macrophagecolony-stimulating factor (20 ng/ml), and transforming growth factor-β (2 ng/ml). In addition, LPS (2 μl/ml), MMP (5 μg/ml), or P. gingivalis PE DHC (20 μg/ml as a sonicated liposome preparation) were added to wells to stimulate IL-6 secretion. Cultures were harvested after 5 days, stimulated in culture for 4 hours with phorbol 12-myristate 13-acetate, ionomycin and brefeldin A and stained for Thy1.2, intracellular IFNγ, and IL-17 and analyzed by fluorescence-activated cell sorting after gating on Thy 1.2+ cells.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

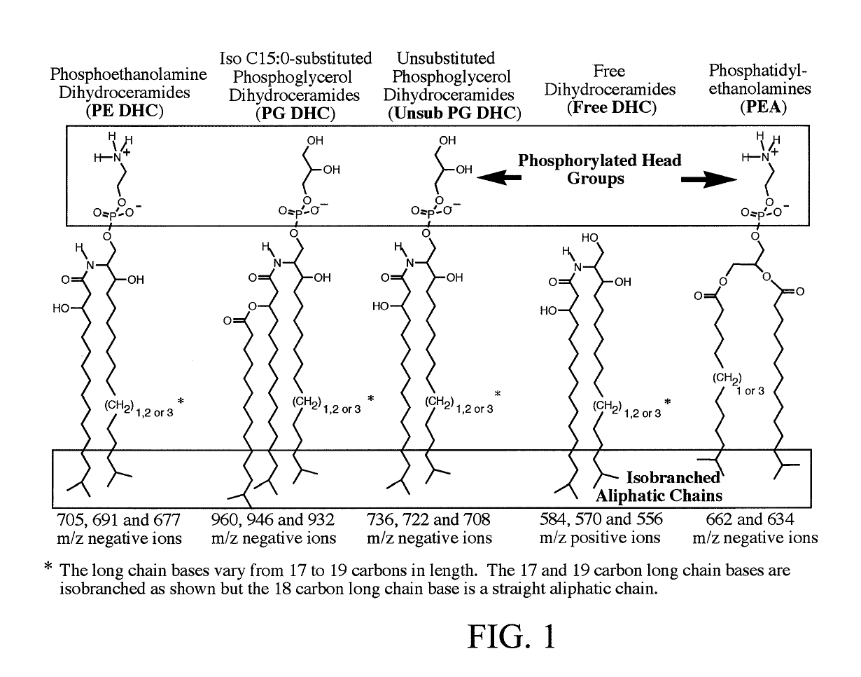

Some embodiments of the present invention utilize in a novel and unexpectedly beneficial way information on bacterial lipids in humans or animals. In particular, some of the embodiments of the present invention utilize information on occurrence of bacterial lipids in a human or an animal in a novel and unexpectedly way that is indicative of an inflammatory or an autoimmune disease or a condition in the human or the animal. Bacterial lipids utilized in the relevant embodiments of the present invention are synthesized by pathologic or non-pathologic bacteria found in a human or an animal organism but not synthesized by the organism itself. These lipids may be referred to as “bacteria-originated” lipids. In one exemplary embodiment, bacteria-originated lipids are bacterial phosphorylated dihydroceramides (PDHCs), biologically active lipids, unique to bacteria, which are capable of promoting inflammatory reactions in human cells in vitro, as described, for example, in Nichols, et al. “Prostaglandin E2 secretion from gingival fibroblasts treated with interleukin-1 beta: effects of lipid extracts from Porphyromonas gingivalis or calculus.”J. Periodontal. Res. 36(3):142-52 (2001), and Nichols, et al. (2004). Two major classes of biologically active lipids are found in PDHCs: phosphoethanolamine dihydroceramide (PE DHC) and phosphoglycerol dihydroceramide (PG DHC) schematically illustrated in FIG. 1. These lipids, integral parts of the bacterial membranes, are likely released upon the death or phagocytosis/endocytosis of the organism. It is to be understood that the term “bacteria originated lipid” or “bacteria originated lipids” are used herein to refer to lipids derived from bacteria, for example, isolated by various isolation techniques, as well as to substantially similar molecules synthesized or generated under laboratory or industrial conditions.

However, the relevant embodiments of the present invention are not intended to be limited by PDHCs. Rather, any lipid can be used in the embodiments of the present invention, as long as the information on their occurrence, used alone or in combination with other information is indicative of an inflammatory or autoimmune disease or conditions. Some of the bacterial lipids used in the embodiments of the present invention may alter the physiology of mammalian lipids, resulting in disease-related alterations in the presence or levels of mammalian lipids in human tissues including the blood. Some embodiments of the present invention utilize bacteria-originated lipids, or lipids comprising structures not produced by mammals, allowing them to be specifically identified in mammalian tissue using various analytical techniques, such as negative ion electrospray mass-spectrometry and multiple reaction monitoring mass-spectrometry (MRM-MS).

As discussed above, bacterial lipids utilized in the methods of the present invention generally originate in bacteria inhabiting human and animal bodies and organisms. Some of these bacterial are habitual inhabitants and are often referred to as “commensal” bacteria, particularly when they are not associated with any pathological states or conditions. Some other of the bacterial are described as “pathological,” particularly if they are typically not found in human or animal organisms, or found in low numbers, and their presence or increased numbers is associated with a pathological state. It is noted that the same bacterial species can be classified as both “commensal” or “pathological,” depending on the accepted classification system, pathology paradigm, bacterial numbers, and other factors. The present invention is therefore not limited to the uses of the lipids originating from commensal, pathological, or any other category of bacteria. Some non-limiting examples of the bacterial lipids used in the methods described herein originate in Bacteroides or Prevotella, Porphyromonas, Tannerella, Prevotella and Parabacteroides genera of bacteria.

One embodiment of the present invention provides a method for detecting an inflammatory or an autoimmune condition, comprising analyzing or detecting bacterial lipids in a sample; and, comparing results of the analysis of the bacterial lipids in the sample with information on occurrence of the bacterial lipids in a comparable sample, wherein the comparison is indicative of the inflammatory or the autoimmune condition. A sample can be obtained from a human or an animal. The method for detecting an inflammatory or an autoimmune condition can further comprise, prior to the step of analyzing, obtaining by any suitable method, such as extracting, a lipid fraction from the sample. The step of analyzing can comprise one or more of: identifying the bacterial lipids; quantitating the bacterial lipids; or determining one or more quantitative relationship among categories of the bacterial lipids detected during the analysis. The information on occurrence of the bacterial lipids can include information on one or more quantitative relationship among categories of the bacterial lipids.

In some of the embodiments, the bacterial lipids analyzed in the method discussed above are PDHCs, including phosphoethanolamine PE DHCs and phosphoglycerol dihydroceramides PG DHCs. In some of the embodiments, the analysis involves determining the ratio of total ion abundance of PG DHC to PE DHC. In one exemplary embodiment, the methods described herein use a ratio PG DHC to PE DHC as indicative of MS. In one example, an increased ratio PG DHC to PE DHC in a blood sample, as compared to a control bloods sample obtained from a non-MS human subject, indicates the presence of MS.

As described herein, bacteria-originated lipids, such as PDHCs, that originate from bacteria found in multiple sites in humans (gingiva, GI tract and vagina), possess previously unknown immunomodulating properties. Accordingly, the present invention encompasses compositions or medicaments comprising bacteria-originated lipids, which are useful for modulating or affecting immune responses, as well as uses and methods of using bacteria-originated lipids to modulate immune responses in a human or an animal. In some exemplary embodiments, compositions, uses and methods induce or exacerbate an autoimmune or an inflammatory state in a human or an animal. Such embodiments can be useful for research or diagnostic purposes, for example, for creation of animal models or for observation of an autoimmune disease flare-up in a patient. However, compositions, uses and methods that decrease or alleviate an autoimmune or an inflammatory state in a human or an animal are also envisioned and fall within the scope of the present invention. According to some embodiments of the present invention, compositions comprising bacteria-originated lipids contain PE DHC. Corresponding methods of use or uses involve PE DHC-containing compositions.

Some other embodiments of the present invention include compositions comprising bacteria-originated lipids, which affect toll-like receptor (TLR) pathways and activities. In one embodiment, compositions according to some embodiments of the present invention comprise a TLR-receptor ligand. Corresponding methods and uses of such compositions are also included in the scope of the present invention. For example, methods of using such compositions to activate a TLR receptor or a TLR receptor signaling pathway or response are included. Methods that involve binding of a TLR ligand disclosed herein to a TLR receptor for research or diagnostic purposes, such detection of a TLR receptor, are also included in the scope of the embodiments of the present invention. The terms “signaling pathway” or “signaling response” are used in reference to biological processes conventional known as “signaling” which generally involve a molecule binding to and activating a protein known as a “receptor”, which, in turn, affects other molecules, thus generating a so-called signaling response, cascade or pathway. The term toll-like receptors (TLRs) is used herein in a conventional manner to refer to a class of proteins that are currently known to play an important role in the innate immune system, and to generally recognize structurally conserved molecules derived from microbes.

The term “composition,” as used herein, encompasses compositions of matter, chemical, analytical, pharmaceutical, therapeutic, preventive or diagnostic compositions, biologically, pharmacologically, immunologically or immunochemically active compositions. The term “composition” also includes medicaments, drugs, medicines, pharmaceuticals, reagents, such as analytical reagents. The term “compositions” encompasses compositions that include one component or ingredient, as well as compositions including more than one component or ingredient. Compositions can comprise both “active” and “inactive” ingredients or components. The term “active” as used herein in reference to a component or ingredient of a composition (which can also be denoted as an “agent”) refers to a compound that possesses an an activity relevant to the use of the composition. As used herein, the term “effective amount” refers to an amount of an active agent that exhibits an activity relevant to the use of the compositions. Effective amounts vary with various uses, durations, other included into the compositions, and other factors. It is to be understood that any of the components of the compositions according to the embodiments of the present invention that are denoted as inactive agents, explicitly or by implication, nevertheless can change the activity of the active agents, and can also have independent effects. The term “method” as used herein encompasses methods of using and uses of compositions according to various embodiments of the present invention.

The terms “detect,” “detecting,” “indicate,” “indicative” and similar terms are used in this document to broadly to refer to a process or discovering or determining the presence or an absence, as well as a degree, quantity, or level, or probability of occurrence of something. For example, the term “detecting” when used in reference to a disease or a condition can denote discovery or determination one or more of presence of a disease or a condition, absence of a disease or a condition, progression, level or severity of a disease or a condition, as well as a probability of present or future exacerbation of symptoms, or of efficacy of a treatments. The foregoing list is not intended to be exhaustive, and the terms “detect,” “detecting,” “indicate,” “indicative” and similar can also refer to other things.

The terms “analysis” or “analyzing” and similar terms are used herein to broadly refer to studying or determining a nature, properties, or quantity of an object under analysis, or its components. Analysis can include detection, as discussed above. Analysis can also involve chemical or biochemical manipulations or steps, as well as manipulations or steps of other nature, as well as manipulation of information in an appropriate manner (for example, storage of information in computer memory and computer calculations may be used).

The term “occurrence” when used in reference to bacterial lipids utilized in some of the embodiments of the present invention is used to denote incidence of the bacterial lipids, as well as frequency of their appearance, quantity, or distribution throughout different classes or subclasses. In some embodiments of the present invention can utilize any of the foregoing information falling within the meaning of the term “occurrence” in relation to one or more bacterial lipids, as well as classes and subclasses of such lipids. Combination of such information on the occurrence of lipids can be referred to as “pattern” or “lipid pattern.” The information on occurrence of bacterial lipids, or lipid patterns, obtained in the course of performing the methods described herein can be compared or correlated with the information previously obtained, processed or stored. The results of such comparison, according to certain embodiments of the present invention, lead to detection of a disease or a condition. When the information on occurrence of bacterial lipids is derived from a sample obtained from a human or an animal patient, the methods is useful for detection of a disease or a condition in the patient. It can be said that the methods of the present invention utilize bacterial lipids, including bacteria-originated lipids, as markers, biomarker, or biological marker to detect a disease or a condition, such as autoimmune or inflammatory disease or condition. In other words, occurrence of bacterial lipids is used in a present invention as a characteristic measured and evaluated as an indicator of certain biological processes. These processes may include autoimmune diseases, such as Rheumatoid Arthritis and Systemic Lupus Erythematosus, and generalized vascular disease, as it occurs in atherosclerosis.

The analysis of bacterial lipids used in the methods of the present invention can involve various analytical techniques suitable for qualitative or quantitative detection of lipids, including, but not limited to HPLC, gas chromatography, mass-spectrometry, immunochemical techniques and assays (ELISA), and lipid arrays (described, for example, in U.S. Patent Publication US20070020691.

The term “condition” when used in reference to the embodiments of the invention disclosed herein is used broadly to denote a biological state or process, such as an immune or inflammatory response, which can be normal or abnormal or pathological. The term “condition” can be used to refer to a medical or a clinical condition, meaning broadly a process occurring in a body or an organism and distinguished by certain symptoms and signs. The term condition can be used to refer to a disease or pathology, meaning broadly an abnormal disease or condition affecting a body or an organism.

Some conditions detected by the detection methods disclosed herein are inflammatory or autoimmune conditions. Non-limiting examples or autoimmune conditions are rheumatoid arthritis, systemic lupus erythematosus (SLE), diabetes (type 1) or multiple sclerosis (MS). Non-limiting examples of inflammatory conditions are periodontal disease or atherosclerosis

As used herein, the terms “multiple sclerosis” or “MS” refer to a disease or condition that affects the brain and spinal cord (central nervous system) of humans and can exhibit any of the symptoms described below. While MS is currently characterized in the medical field as a condition arising out of autoimmune damage to the myelin sheath, the embodiments of the present invention are not limited by this characterization and encompass detection of MS-like diseases and conditions that are broadly encompassed by the clinical criteria described below, even if these diseases and conditions have causes, origins or mechanisms different from those covered by the presently accepted MS paradigm. MS is most commonly diagnosed between ages 20 and 40, but can be observed or diagnosed at any age. MS symptoms vary, and the location, severity and duration of each MS attack can be different. Episodes can last for days, weeks, or months and alternate with periods of reduced or no symptoms, generally referred to as remissions. It is common for MS to relapse, but it also may continue without periods of remission. MS patients can have any of the following symptoms, in various combinations: muscle symptoms, which include loss of balance, muscle spasms, numbness or abnormal sensation in any body area, problems moving arms or legs, problems walking, problems with coordination and making small movements, tremor in one or more arms or legs or weakness in one or more arms or legs; bowel and bladder symptoms, which include constipation and stool leakage, difficulty beginning to urinated, frequent need to urinate, strong urge to urinate, urine leakage (incontinence), eye symptoms, which include double vision, eye discomfort, uncontrollable rapid eye movements, vision loss (usually affects one eye at a time); numbness, tingling, or pain; facial pain; painful muscle spasms; tingling, crawling, or burning feeling in the arms and legs; other brain and nerve symptoms, which include decreased attention span, poor judgment, and memory loss, difficulty reasoning and solving problems, depression or feelings of sadness, dizziness and balance problems, hearing loss; sexual symptoms; speech and swallowing symptoms, which include slurred or difficult-to-understand speech, trouble chewing and swallowing; fatigue is also one of the symptoms.

The terms “sample” or “samples,” as used interchangeably herein, refer to any cell or tissue samples or extracts originating from human or animal subject, and include samples of human or animal cells or tissues as well as cells of non-human or non-animal origin, including bacterial samples. A sample can be directly obtained from a human or animal organism, or propagated or cultured. Samples can be subject to various treatment, storage or processing procedures before being analyzed according to the methods described herein. Generally, the terms “sample” or “samples” are not intended to be limited by their source, origin, manner of procurement, treatment, processing, storage or analysis, or any modification. Samples include, but are not limited to samples of human cells and tissues, such as blood samples, cerebrospinal fluid samples, synovial tissue samples, synovial fluid samples, brain tissue samples, blood vessel samples, or tumor samples. Samples encompass samples of healthy or pathological cells, tissues or structures. Samples can contain or be predominantly composed of bacterial cells. The terms samples or samples can refer to the samples of structures or buildup commonly referred as plaques, such as atheromatous plaque, dental plaque, senile plaque, mucoid, dermal plaque. Some examples of samples are plasma samples, including the samples from periodontally healthy subjects, blood plasma samples from subjects with generalized severe destructive periodontal disease, such as chronic periodontitis, subgingival microbial plaque samples, carotid atheroma samples and tissue samples derived from human brain. Some other examples of samples are samples of teeth, skin, or kidneys.

In one of its embodiments, the present invention provides a lipid-specific antibody capable of specific binding to a PDHC lipid category, such as an antibody capable of specific binding with PG DHC or PE DHC. Antibodies described herein are useful for detecting a PDHC lipid in a sample, for modulating an immune response in a human or animal cell or tissue or in a human or an animal organism, and can be incorporated into pharmaceutical compositions and medicaments for modulating immune responses. Antibodies described herein can also be useful in diagnostic methods, such as detection methods according to some other embodiments of the present invention described herein. Antibodies described herein are also useful for detecting a PDHC lipid in a sample and can be incorporated into diagnostic kits and reagents.

The terms “modulating,” “modulation” and similar terms, when used in reference to immune responses and pathways (which can also be denoted as “immunomodulating”), inflammatory responses and pathways, as well as TLR responses and pathways are used generally to refer to modification of immune responses, processes and cascades in response to a modulating agent, such as an antibody. Immunomodulation can result in an increased immune response or a decreased immune response, or both an increase and a decrease, when assessed through different parameters or processes. The term “immune response” encompasses the whole scope of animal immune response, including innate and adaptive immunity.

The composition according to some embodiments of the present invention can be readily formulated with, prepared with, or administered with, a pharmaceutically acceptable carrier. Such preparations may be prepared by various techniques. Such techniques include bringing into association active components of the compositions and an appropriate carrier. In one embodiment, compositions are prepared by uniformly and intimately bringing into association active components of the compositions with liquid carriers, with solid carriers, or with both. Liquid carriers include, but are not limited to, aqueous formulations, non-aqueous formulations, or both. Solid carriers include, but are not limited to, biological carriers, chemical carriers, or both.

The compositions according to some embodiments of the present invention may be administered in an aqueous suspension, an oil emulsion, water in oil emulsion and water-in-oil-in-water emulsion, and in carriers including, but not limited to, creams, gels, liposomes (neutral, anionic or cationic), lipid nanospheres or microspheres, neutral, anionic or cationic polymeric nanoparticles or microparticles, site-specific emulsions, long-residence emulsions, sticky-emulsions, micro-emulsions, nano-emulsions, microspheres, nanospheres, nanoparticles and minipumps, and with various natural or synthetic polymers that allow for sustained release of the composition including anionic, neutral or cationic polysaccharides and anionic, neutral cationic polymers or copolymers, the minipumps or polymers being implanted in the vicinity of where composition delivery is required. Polymers and their use are described in, for example, Brem et al, Journal of Neurosurgery 74:441-446 (1991). Furthermore, the active components of the compositions according to some embodiments of the present invention can be used with any one, or any combination of, carriers. These include, but are not limited to, anti-oxidants, buffers, and bacteriostatic agents, and may include suspending agents and thickening agents.

For administration in a non-aqueous carrier, active components of the compositions according to some embodiments of the present invention may be emulsified with a mineral oil or with a neutral oil such as, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, soybean oil, canola oil, palm oil, olive oil and myglyol, wherein the number of fatty acid carbons is between 12 and 22 and wherein the fatty acids can be saturated or unsaturated. Optionally, charged lipid or phospholipid can be suspended in the neutral oil. More specifically, use can be made of phosphatidylserine, which targets receptors on macrophages. Use can be made of active components of the compositions according to embodiments of the present invention formulated in aqueous media or as emulsions using techniques known to those of ordinary skill in the art.

The compositions according to some embodiments of the present invention can comprise active agents described elsewhere in this document, and, optionally, other therapeutic and/or prophylactic ingredients. The carrier and other therapeutic ingredients must be acceptable in the sense of being compatible with the other ingredients of the composition and not deleterious to the recipient thereof.

The compositions according to some embodiments of the present invention are administered in an amount effective to induce a therapeutic response in an animal, including a human. The dosage of the composition administered will depend on the condition being treated, the particular formulation, and other clinical factors such as weight and condition of the recipient and route of administration. In one embodiment, the amount of the composition administered corresponds from about 0.00001 mg/kg to about 100 mg/kg of an active component per dose. In another embodiment, the amount of the composition administered corresponds to about 0.0001 mg/kg to about 50 mg/kg of the active component per dose. In a further embodiment, the amount of the composition administered corresponds to about 0.001 mg/kg to about 10 mg/kg of the active component per dose. In another embodiment, the amount of the composition administered corresponds to about 0.01 mg/kg to about 5 mg/kg of the active component per dose. In a further embodiment, the amount of the composition administered corresponds to from about 0.1 mg/kg to about 1 mg/kg of the active component per dose.

Useful dosages of the compounds of the present invention can be determined by comparing their in vitro activity, and in vivo activity in animal models. Methods for the extrapolation of effective dosages in mice, and other animals, to humans are known in the art; for example, see U.S. Pat. No. 4,938,949.

Modes of administration of the compositions used in the invention are exemplified below. However, the compositions can be delivered by any of a variety of routes including: by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository or aerosol spray. Routes of administration include, but are not limited to, topical, intradermal, intrathecal, intralesional, intratumoral, intrabladder, intravaginal, intra-ocular, intrarectal, intrapulmonary, intraspinal, dermal, subdermal, intra-articular, placement within cavities of the body, nasal inhalation, pulmonary inhalation, impression into skin and electroporation.

Depending on the route of administration, the volume of a composition according to some embodiments of the present invention in an acceptable carrier, per dose, is about 0.001 ml to about 100 ml. In one embodiment, the volume of a composition in an acceptable carrier, per dose is about 0.01 ml to about 50 ml. In another embodiment, the volume of a composition in an acceptable carrier, per dose, is about 0.1 ml to about 30 ml. A composition may be administered in a single dose treatment or in multiple dose treatments, on a schedule, or over a period of time appropriate to the disease being treated, the condition of the recipient and the route of administration. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

### EXAMPLES

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

Procurement, Storage and Processing of Bacterial Samples

Bacterial samples previously stored frozen at −80° C. in skim milk were grown on blood agar plates after demonstrating purity of bacterial isolates. Bacteria were identified by 16 S rRNA sequencing (˜1400 bp). Phenotypic tests were done when needed to fully identify an organism. The plates were scraped to recover the bacterial colonies and were extracted using the phospholipid extraction procedure described in Bligh, E. G. & Dyer, W. J. “A rapid method of total lipid extraction and purification.” Can. J. Biochem. Physiol. 37: 911-917 (1959), as modified by the procedures described in Garbus, J. et al. “Rapid incorporation of phosphate into mitochondrial lipids” J. Biol. Chem. 238:59-63 (1968). Porphyromonas gingivalis (type strain, ATCC#33277), Tannerella forsythia and Prevotella intermedia (VPI 8944) were grown in broth culture and after pelleting bacteria by centrifugation, the bacterial pellets were stored frozen until processing. P. gingivalis and P. intermedia were grown in broth culture according to the procedures described, for example, in Nichols et al. “Release from monocytes treated with lipopolysaccharides isolated from Bacteroides intermedius and Salmonella typhimurium: Potentiation by gamma interferon” Infect. Immun. 59:398-406 (1991), and Nichols, F. C. & Rojanasomsith, K. “Porphyromonas gingivalis lipids and diseased dental tissue”. Oral Microbiol. Immunol. 21:84-92 (2006). At the time of lipid extraction, samples of bacterial pellets were removed and extracted using the phospholipid extraction procedure discussed above.

Procurement, Storage and Processing of Human Samples

Human tissue and blood samples were obtained according to conventional procedures and guidelines. All tissue and blood samples were stored frozen until processing. Human tissue samples were stored frozen (−20° C.) until the time of lipid extraction. Atheroma samples were processed as follows. The patent segment of the common carotid artery (control samples) was excised from the grossly apparent atheroma of the carotid body, and PDHCs in the lipid extracts from the individual paired samples were quantified. The patent carotid artery samples showed no apparent gross atheroma formation though these artery segments were partially calcified within the artery wall. Gingival tissue, atheroma and brain samples were thawed and at least 20 mg of tissue was minced and extracted for several days in organic solvent according the method of Bligh & Dyer (1959). After drying organic solvent extracts under nitrogen, the lipid extracts were reconstituted in hexane:isopropanol:water (HPLC solvent, 6:8:0.75, v/v/v), vortexed and centrifuged. The resultant supernatants were recovered, a sample of defined volume (5 μl) was dried and weighed, and a defined amount of each sample was transferred to a clean vial either for further processing or for MRM-MS analysis. For brain samples, 10 mg of each lipid extract was fractionated by normal phase HPLC as described in Nichols et al. (2004). The fractions expected to contain the PDHC lipids were pooled and dried. Each brain lipid isolate was then reconstituted in 300 μl of HPLC solvent and 5 μl was analyzed by MRM-MS for the bacterial lipids of interest. For each subgingival plaque sample, 50 μl of lipid extract was dissolved in 200 μl of HPLC solvent and 5 μl of each sample was analyzed by MRM-MS. For gingival tissue samples, 1 mg of lipid extract was dissolved in 300 μl of HPLC solvent and 5 μl of each sample was analyzed by MRM-MS. Citrated blood samples, obtained by venipuncture from periodontal patients, were diluted 2:1 (v/v) in saline and subjected to Ficoll-Hypaque centrifugation. Plasma samples were aspirated following centrifugation and stored frozen until lipid extraction. For lipid extraction, the plasma samples were thawed and 0.5 ml of each sample was extracted for lipids as described above. The dried lipid samples were reconstituted in 300 μl of HPLC solvent and analyzed by MRM-MS.

Analysis of Lipid Samples

Individual lipid samples were analyzed using a 4000 QTrap 4000 mass spectrometer (AB Sciex®, Foster City, Calif.). A standard volume of each lipid sample (5 μl) was analyzed by flow injection and HPLC solvent was run at a rate of 80 μl/min. Using previously purified lipid preparations of each phosphorylated dihydroceramide class, the instrument parameters were optimized for detection of each lipid component based on gas phase transitions depicted in FIG. 1. Standard curves were generated using serially diluted lipid standards of known quantity and linearity of lipid quantification was observed (regression coefficients >0.99). In addition, carryover of individual lipid ion transitions into other monitored transitions was not observed. Using the optimized instrument parameters, each lipid extract from tissue, blood and bacterial samples was individually analyzed. Each lipid ion transition peak was electronically integrated and the percentage abundance of each lipid class was calculated from the integrated lipid ion transition peaks. For each category of tissue or blood samples, all samples within a particular tissue or blood category were analyzed during a single analysis session. Two-factor ANOVA or the paired student t test was used to test for significant differences between sample categories.

Mice

Female C57BL/6 (WT) mice were obtained from Jackson Labs (Bar Harbor, Me.). TLR2^{-/-} mice were a generous gift of Dr. S. Akira (Osaka University, Japan), IL-15^{-/-} mice and IL-15Rα^{-/-} mice were a generous gift from Dr. Leo LeFrancois (University of Connecticut Health Center). All mice were maintained and bred in accordance with conventional animal care procedures.

Induction of Experimental Allergic Encephalomyellites (EAE)

EAE served as a murine model of MS. Female mice (4-8 weeks old) were immunized with 100-200 μm of myelin oligodendrocyte glycoprotein peptide (35-55) (MOG) emulsified with CFA (containing 500 μg of H37RA mycobacteria) (DIFCO Co-BD Diagnostics, Sparks, Md.) via a subcutaneous (s.c.) injection on Day 0. 200-250 ng of Pertussis toxin (List Biologicals Labs, Campbell, Calif.) was injected intravenously (i.v.) on Day 0 and again on Day 2. In addition, mice were injected intraperitoneally (i.p.) on Day 0 with either P. gingivalis lipid or the vehicle control, 70% ethanol (EtOH). EAE was scored as: Grade 1-tail paralysis; Grade 2-weakness of hind limbs with an altered gait; Grade 3-hind limb paralysis; Grade 4-front limb paralysis; Grade 5-death.

Purification and Verification of P. Gingivalis Lipids

P. gingivalis (ATCC#33277, type strain) was grown and lipids extracted and fractionated by HPLC as previously described in Nichols et al. (2004); Nichols “Novel ceramides recovered from Porphyromonas gingivalis: relationship to adult periodontitis” J. Lipid Res. 39:2360-2372 (1998). HPLC fractions highly enriched for PE DHC lipids were identified via electrospray-MS using a Micromass Quattro II mass spectrometer system as described in Nichols et al. (2004). HPLC fractions containing highly enriched PE DHC lipids were pooled and each combined fraction was verified to be of greater than 95% purity by electrospray-MS.

Processing of Lipids for Administration to Animals and Addition to Tissue Culture

For treatment of mice, preweighed lipids were dissolved in 70% ethanol to achieve a final concentration of 125 ng/μl, and sonicated for 2.5 minutes immediately before injection into experimental animals. This preparation was also used for drying lipids onto tissue culture wells. For direct addition to cell cultures, the lipids were dissolved in culture medium at 125 ng/μl and sonicated for 2.5 minutes to produce a liposome preparation for administration to cells in culture.

Derivation and Stimulation of Bone Marrow Dendritic Cells (DCs)

Bone marrow cells from C57BL/6 and TLR2^{-/-} mice were cultured at 2×10⁵ cells/ml in RPMI containing 10% FCS, 2-ME, and 20 ng/ml recombinant murine GM-CSF for 9 days. Bone marrow DCs (BMDCs) were harvested at Day 9 and were greater than 80% CD11c+. LPS (1 μg), MMP (10 μg) (a bacterial lipoprotein and known TLR-2 ligand: Palmitoyl-Cys ((RS)-2,3-di(palmitoyloxy)-propyl)-Ser-Ser-Asn-Ala-OH(pam3-Cys-Ser-Ser Asn-Ala-OH) (Bachem H-9460), PE DHC (2.5 μg) or 70% EtOH, all in 20 ul volumes, were allowed to dry in the wells of a 24-well plate overnight prior to the addition of BMDCs. BMDCs were cultured in the ligand-bound 24-well plates at 1×10⁶ cells/ml in RPMI containing GM-CSF. After 18 hrs, culture supernatants were harvested and tested for IL-6 via ELISA.

In Vitro Generation of Th17 T Cells

CD4+CD25− T cells (Teff) were derived from WT mice using magnetic bead purification (Miltenyi Biotec, Auburn, Calif.). T cell-depleted splenocytes (Tds) were derived from WT or TLR2^{-/-} mice using magnetic bead purification followed by irradiation (2600R). Teff (0.25×10⁶/well) and Tds (0.75×10⁶/well) were cultured in 24-well plates with anti-CD3 antibody (1 μg/ml), GM-CSF (20 ng/ml) (Pierce Inc., Thermo Fisher Scientific, Rockford, Ill.) and recombinant TGF-β (2 ng/ml) (R&D). In addition, LPS (2 μg/ml), MMP (5 μg/ml) or P. gingivalis PE DHC (20 μg/ml of sonicated liposome preparations) were added to wells to stimulate the secretion of IL-6. Cultures were harvested after 5 days, stimulated in culture for 4 hrs with phorbol myristyl acetate and ionomycin and stained for Thy1.2, intracellular IFNγ, and IL-17 and analyzed by FACS after gating on Thy 1.2+ cells.

Derivation and Phenotypic Analysis of Spinal Cord-Derived Mononuclear Cells

Spinal cord mononuclear cells were derived as previously described in Korn et aL“Myelin-specific regulatory T cells accumulate in the CNS but fail to control autoimmune inflammation” Nat. Med. 13:423-431 (2007) and stained for CD4 (FITC α-CD4 (GK1.5) BD Pharmingen) and Foxp3 (APC α-FoxP3 (HK-16s; E-Bioscience), or stimulated in culture for 4 hrs with phorbol myristyl acetate and ionomycin prior to staining for Thy1.2 (PE-Cy7 anti-CD 90.2; E-Biosciences) and intracellular IFNγ (APC αIFNγ; BD Pharmingen) and IL-17 (Alexa Fluor 488 α-IL-17A; BD Pharmingen).

Recovery of Bacterial Lipids from the Brains of Mice with EAE

Mice treated with PBS, EtOH or with 25 ng, 250 ng, or 2.5 μg PE DHC were sacrificed after day 20 post-EAE immunization. The brains were removed and extracted for phospholipids according to the method of Bligh and Dyer as previously described in Nichols “Distribution of 3-hydroxy iC17:0 in subgingival plaque and gingival tissue samples: Relationship to adult periodontitis” Infect. Immun. 62:3753-3760 (1994). Lipid extracts were dissolved in hexane isopropanol:water (6:8:0.75, v/v/v/) and three 0.5 mg aliquots were dispensed into glass tubes supplemented with 30 ng of isobranched C_{20:0}. Lipid samples were hydrolyzed for 4 hours in 2N KOH, acidified and fatty acids extracted into chloroform and dried. Lipids were treated to form pentofluorobenzyl ester, trimethylsilyl ether derivatives and analyzed by negative ion chemical ionization GC-MS, as described in Nichols (1994). Fatty acid recovery was quantified by selected ion monitoring for characteristic fatty acid negative ions. The data were expressed as picograms of 3-OH isobranched (iso)C_{17:0} per 0.5 mg of total brain lipid extracted.

Statistical Procedures for EAE Animal Model Studies

The cumulative disease index (CDI) was obtained by summing the daily average disease scores through Day 20. A mean of these daily disease scores (Mean Daily Disease) (+/−SEM) was calculated based on the 20 days of observation The Mean Daily Disease scores were compared using the Wilcoxin Signed Rank tests for two samples. Disease incidence frequencies were compared using Chi square analysis. Values for mean maximum severity of EAE were compared using the Wilcoxin Signed Rank test. Values for mean day of onset of EAE were compared using the Student's t-test. For analysis of spinal cord populations, percentages were compared using Student's t test. Bacterial fatty acid levels in brain lipid extracts for each treatment group were evaluated using least squares linear regression analysis that included calculation of correlation coefficients. For each dose of bacterial lipid administered, linear regression analysis compared the final EAE score with the mean bacterial fatty acid recovered per 0.5 mg of brain lipid extract. The mean bacterial fatty acid levels were calculated from three replicate brain lipid determinations.

### Example 1

### Lipid Analysis of Bacterial Species from Human Isolates

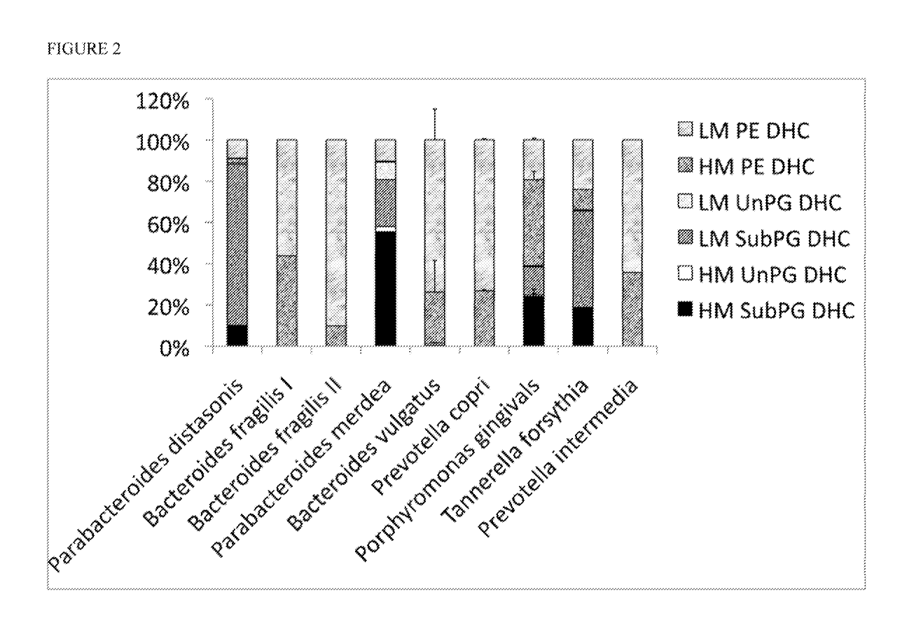

Lipid extracts from 95 intestinal bacterial species from a total of 247 individual human isolates were analyzed. The results of the analysis are schematically represented in FIG. 2. As illustrated in FIG. 2, the lipid analysis revealed that these species varied in their capacity to produce either PE DHC or PG DHC and also varied in their production of the high mass (HM) versus the low mass (LM) forms of these PDHCs. For example, the PDHC lipid constituents produced by P. gingivalis were predominantly HM PE DHC lipids whereas T forsythia produces primarily LM PG DHC forms.

The lipid analysis of the intestinal and oral bacterial species demonstrated that different strains of the same intestinal species may produce PDHCs with different levels of PG DHC or PE DHC. Intestinal bacteria assessed in the analysis exhibited a tendency to produce primarily PE DHC or PG DHC, but not both. Of the intestinal and periodontal organisms observed to produce PG DHCs, only B. merdea produced a small amount of the unsubstituted (“UnPG DHC”) lipids (˜11% of total PDHC), whereas the remaining intestinal and oral bacteria produced negligible amounts of UnPG DHC lipids.

The lipid analysis of the intestinal and oral bacterial species showed that they varied in their capacity to produce specific PDHC lipids, and that the combinations of intestinal and oral bacterial organisms have the ability to deposit unique mixtures of PDHCs in human tissues.

### Example 2

### Lipid Analysis of Human Samples

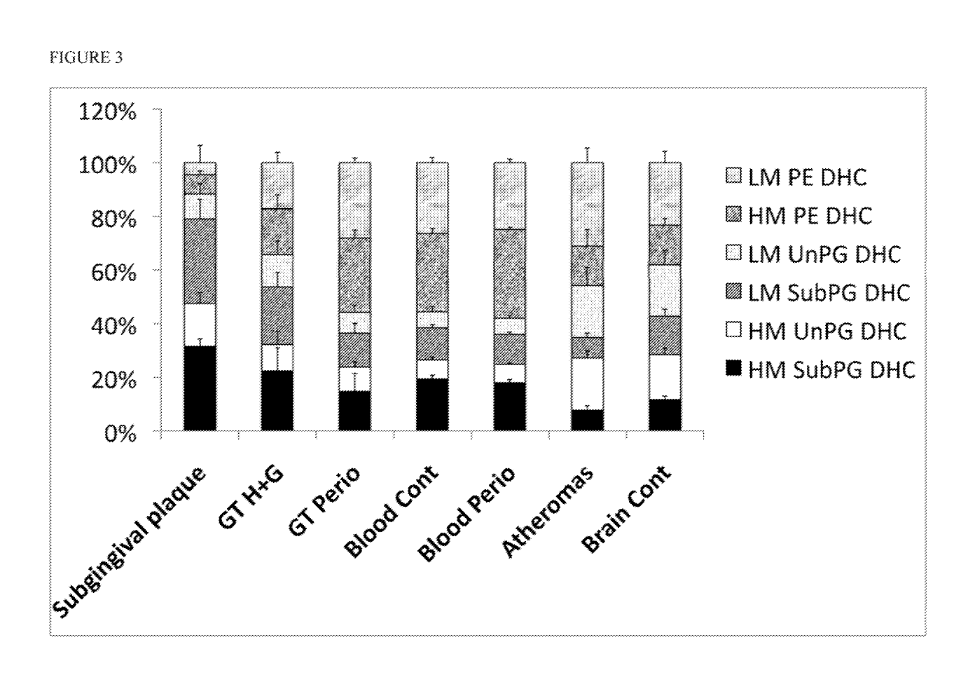

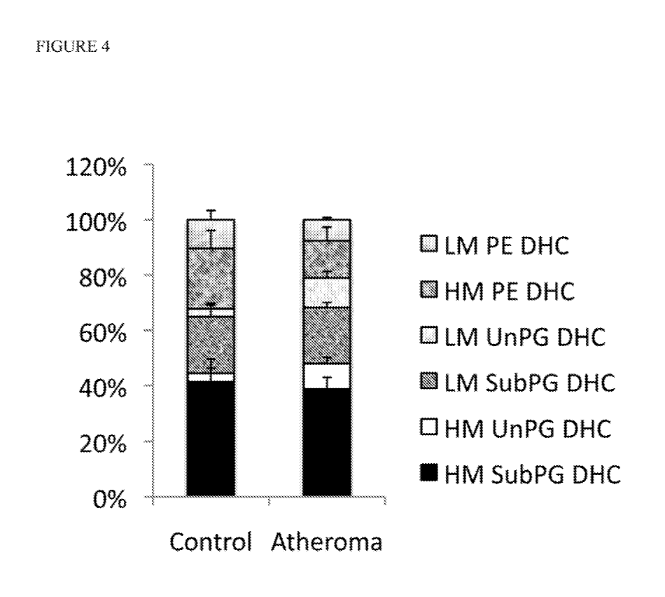

The results of the lipid analysis of human samples are schematically represented in FIGS. 3 and 4. The following samples obtained from human subjects were analyzed: subgingival plaque samples (2 samples) healthy/mildly inflamed gingival tissue (GT H+G, 7 samples), periodontitis gingival tissue samples (GT Perio, 6 samples), control blood plasma samples from periodontally healthy subjects (Blood Cont, 8 samples), blood plasma from patients with generalized severe periodontitis (Blood Perio, n=7), carotid atheroma (Atheroma, n=11) and postmortem brain samples from non-MS subjects. Deposition of PDHCs was observed in all of the human tissue samples examined. The distribution of PDHCs in the examined tissue samples showed distinctive patterns. PDHCs detected in human tissue samples were a mixture of HM and LM forms and revealed significant percentages of both LM or HM UnPG DHC lipids. Comparative analysis of blood plasma samples from periodontally healthy subjects and subjects with chronic periodontitis revealed substantial percentages of both LM or HM UnPG DHC lipids. Analysis of lipid extracts from atheroma artery segments revealed higher percentages of RM or LM UnPG DHC, when compared with the control artery extracts. The total ion abundances of PDHC lipids per μg of total lipid extract were 33 times higher on average in the control artery segments than the atheroma segments. Lipid extracts of brain samples showed a mean percentage of UnPG DHC lipids comparable to or higher than those observed in carotid atheromas. In contrast, subgingival microbial plaque samples taken from gingival crevices at periodontitis sites showed only minimal levels of UnPG DHC. Comparative analysis of PDHC lipids in healthy versus inflamed (periodontitis) gingival tissue and associated blood plasma samples was performed. Two-factor ANOVA revealed significantly lower percentages of HM and LM SubPG DHC lipids and significantly higher percentages of HM and LM PE DHC lipids in periodontitis gingival tissue samples versus healthy samples. Similarly, blood plasma samples demonstrated a significant increase in the percentage of HM PE DHC lipids in periodontitis plasma versus healthy plasma samples, while SubPG DHC percentages were not lower in plasma samples from periodontitis patients. The analysis showed that shifts in the deposition of specific bacterial lipids (PE DHCs) in gingival tissues was directly correlated with expression of destructive periodontal disease and that this specific increase in PE DHC is also reflected in blood plasma levels. In control carotid samples, atheroma samples, and in brain samples, the percentages of PG DHC lipids were relatively higher than that in both blood samples and diseased gingival. Analysis of deposition of PDHC in human tissues showed that distribution patterns of bacterial PDHCs in human tissues correlate with health and disease states. In particular, distribution patterns of bacterial PDHCs in human tissues correlate with inflammation states.

### Example 3

### Analysis of Brain Tissue Samples from MS Patients with Active Disease

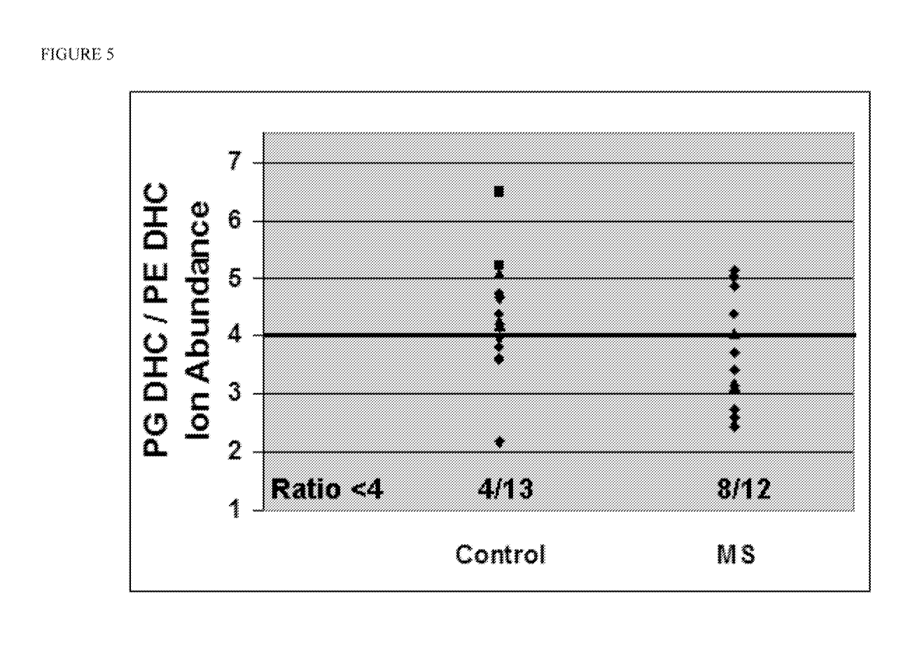

Analysis of coded (blinded) frozen brain samples obtained from control subjects and from MS patients with active disease was performed. The results of the analysis are schematically represented in FIG. 5, which shows the PG DHC/PE DHC ion abundance ratios of 13 control and 12 active MS brain samples. The samples were analyzed for the presence of bacteria-originated PE DHC and PG DHC lipids using MRM-MS. The MRM-MS approach was somewhat different from the MRM-MS approach utilized in the study described in Example 2. Following analysis of the samples by MRM-MS, the ratio of PG DHC to PE DHC, measured as total ion abundance, was calculated. It was observed that all brain samples analyzed contained some level of PDHCs. Quantification of the different PDHC classes from control and active MS patients demonstrated surprising results. While the absolute levels of PE DHC and PG DHC were not statistically different between control and active MS patients (using two factor ANOVA), the proportional recovery of these fractions was different. A higher level of PE DHC together with a slightly lower (or unchanged) level of PG DHC was found in brain samples from active MS patients versus controls (both healthy and other neurological disease (OND) patients), resulting in an MS-specific PDHC lipid pattern. A decrease in mean PG DHC/PE DHC ratios was thus observed in MS brain samples. While the decrease in mean PG DHC/PE DHC ratios did not reach statistical significance, only 31% of control samples (4/13), but 67% of MS samples (8/12), showed a PG DHC/PE DHC ratio of less than 4.0 using this specific analytic approach. While mean ratios differed between control and MS brain samples at the p=0.09 level (unpaired Student's t-Test), one control sample was an outlier, demonstrating a ratio of 2.18 (over 2.2 standard deviations from the mean). If this outlier was removed, the mean brain PG DHC/PE DHC ratios for MS versus controls differed significantly, with p=0.02 (unpaired Student's t-Test). The lipid analysis of brain samples from MS patients described in this example showed that the presence of MS in a patient correlated with a decrease in the PG DHC/PE DHC ratio, measured as ion abundance or mean ratio.

### Example 4

### Analysis of Serum Samples from MS Patients with Active Disease

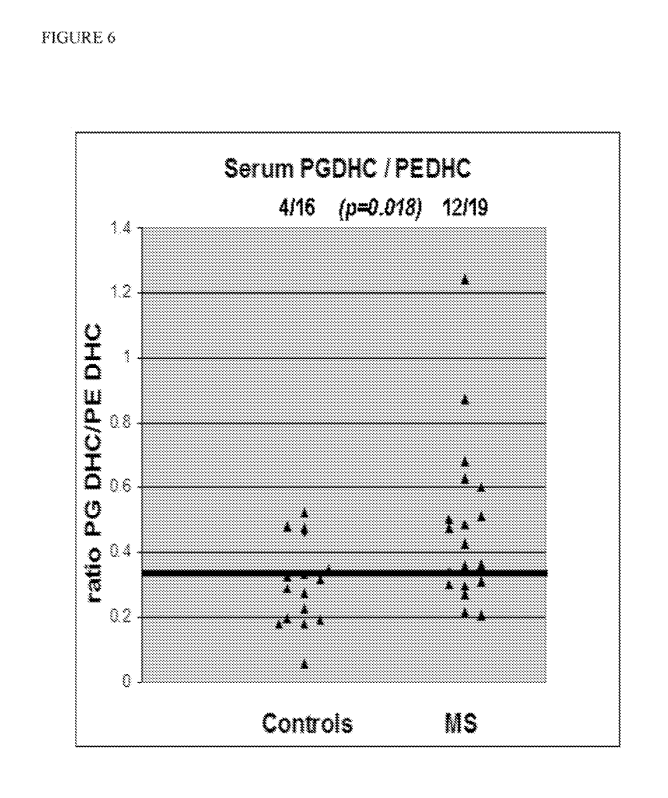

The results of PDHC analysis of serum samples from MS patients with active disease is schematically illustrated in FIG. 6. Serum samples were obtained from a group of healthy control patients and from a group of MS patients (“MS samples”). The MS patients included both genders, a wide age distribution, and represented different MS subtypes and therapeutic treatments. Control samples were obtained from patients that had no acute or chronic health problems, included both genders, and had an age distribution substantially similar to the group of MS patients. Lipids were extracted from the samples and analyzed for the presence of bacteria-originated PE DHC and PG DHC using MRM-MS. In the studies described in this example, as compared to those described in Example 2, serum rather than plasma samples were examined. The analysis of the serum samples involved a somewhat different MRM-MS approach than the approach used in the studies described in Example 2. A group of 19 MS and 16 control samples was analyzed for levels of PDHCs. Ratios of PG DHC to PE DHC total ion abundance were used to compare PDHCs in control vs. MS samples. Statistically significant differences (using several statistical approaches) were found between PDHC levels in control and MS samples. PE DHC levels were decreased, PG DHC levels were similar, and PG DHC/PE DHC ratios were increased in MS versus control samples. Using two factor ANOVA, it was found that the mean absolute ion abundance of PE DHCs (per 5 μg of total serum lipid extract) was statistically significantly lower in MS patients (mean=46,159+/−SEM of 11,360) than in controls (mean=71,684+/−SEM of 7,276). Thus, the absolute amount of PE DHC per 5 μg of total serum lipid extract was significantly lower in MS versus control samples using both Scheffe contrasts among pairs of means (p<0.05) and Fisher LSD (p=0.0006). The mean level of total lipids derived from control serum samples was not significantly different from MS serum samples. Serum PG DHC levels were not significantly different between MS and control samples; thus PG DHC levels served as an “internal reference” for shifts in PE DHC levels. Using this “internal reference,” it was discovered that PG DHC/PE DHC ratios were significantly different between control and MS serum samples. Mean PG DHC/PE DHC ratios were significantly higher in MS serum (0.478+/−SEM of 0.058) versus control serum (0.300+/−SEM of 0.033) (p=0.018, unpaired Student's t-Test). Furthermore, 63% of MS samples (12/19) had ratios greater than 0.35 while only 25% of control patients (4/16) had ratios greater than 0.35 (see FIG. 6). The results showed no obvious correlation with gender, age, MS subtype, or treatment. The test results using serum PG DHC/PE DHC ratios yielded a diagnostic sensitivity of 63% and a specificity of 75% for MS versus controls.

### Example 5

### PDHC Ratios Correlate with the Presence of MS

The comparative analysis of the brain and serum samples described in the prior examples revealed that PE DHC levels are decreased and PG DHC/PE DHC ratios increased in MS sera samples as compared to the control samples, while the reverse pattern was observed in the MS brain samples as compared to the control samples. The experimental results described in the prior examples showed that distribution of PDHCs in tissues and organs, such as blood and brain, correlated with the presence of MS.

### Example 6

### Bacteria-Originated Lipid Patterns in Human Tissues Indicating Autoimmune or Inflammatory Disease or Condition in a Patient

Tissue samples, such as serum samples, are obtained from patients suffering from an inflammatory condition and/or an autoimmune disease. Analysis of the samples for bacteria-derived lipids, such as PE DHCs, is performed. One of the approaches used in the analysis is MRM-MS, which is capable of specific identification and quantification of the lipid families. The distribution patterns of bacteria-derived lipids in the sample are determined and correlated with one or more of the presence of a disease, the stage or activity of the disease, the efficacy of treatment of the disease. The analysis involves assessments of sub-sets samples taking into account one or more of such factors as gender; age; stage and clinical symptoms of the disease, or treatment status of a patient. Reasonably matched control subjects are used. The analysis reveals patterns of bacteria-originated lipids correlating with presence and status of autoimmune or inflammatory disease or condition in a patient. The patterns are used as diagnostic patterns indicative of an autoimmune or inflammatory disease or condition.

### Example 7

### Bacteria Lipid and Population Patterns Indicating Autoimmune or Inflammatory Disease or Condition in a Patient

Samples of commensal intestinal and oral bacteria are obtained from patients suffering from an inflammatory or an autoimmune disease. Bacterial samples are stored and/or cultured as appropriate to obtain sufficient quantity of bacterial for lipid analysis. Analysis of the bacteria-derived lipids, such as PE DHCs, is performed. One of the approaches used in the analysis is MRM-mass spec, which is capable of specific identification and quantification of the lipid families. The distribution patterns of bacteria-derived lipids in the sample are determined and correlated with one or more of the presence of an autoimmune disease in a patient, the stage or activity of the disease, the efficacy of the treatment of the disease. The analysis involves assessments of sub-set samples taking into account one or more of such factors as gender; age; stage and clinical symptoms of an autoimmune disease, or treatment status of a disease. Reasonably matched control subjects are used. The analysis reveals patterns of bacterial lipids and populations correlating with presence and status of autoimmune or inflammatory disease or condition in a patient. The patterns are used as diagnostic patterns indicative of an autoimmune or inflammatory disease or condition.

### Example 8

### Lipid-Specific Antibodies

Lipid-specific antibodies are prepared that specifically react with various PDHC lipid families (PG DHC and PE DHC). Lipid-specific monoclonal antibodies are prepared as follows: PG DHC and PE DHC are conjugated to immune carriers, such as KLH. Mice are immunized with the resulting conjugates. The sera obtained from the immunized mice are tested by ELISAs for binding to PE DHC and PG DHC which have been conjugated to an irrelevant protein carrier. When the sera are positive, splenocytes from the corresponding mice are fused to an appropriate tumor line to generate hybridoma that secrete antibodies to PE DHC or PG DHC. These (uncloned) hybridoma are tested for binding in the ELISA as above, followed by cloning (by limiting dilution) of any hybridoma showing positive antibodies in the ELISA. These subclones are tested for secretion of antibodies that bind either PE DHC or PG DHC, but not both lipids. Continued subcloning of the hybridomas is conducted as necessary to obtain hybridomas that secrete antibodies binding either PE DHC or PG DHC, but not both lipids. Lipid-specific antibodies are used in immunochemical assays, such as ELISA, to rapidly and easily test serum samples for the presence of lipids of interest.

### Example 9

### P. Gingivalis Total Phosphorylated Dihydroceramides Lipids, and Specifically the PE DHC Fraction, Enhanced EAE

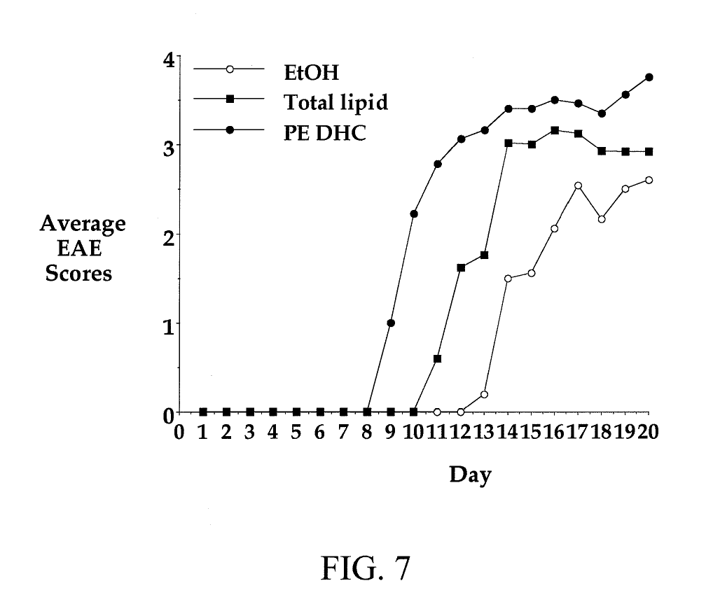

EAE was induced in female C57BL/6 (WT) mice and these mice were also injected i.p. on Day 0 with either P. gingivalis lipid or the vehicle control, 70% ethanol (EtOH). To most effectively detect effects of P. gingivalis lipids in the development of autoimmunity, less severe EAE was induced by using CFA with higher concentrations of H37RA mycobacteria (500 μg/mouse). The effect of administering the P. gingivalis total phosphorylated dihydroceramide lipids (TL) on EAE in wild-type mice was examined. A single i.p. injection of 2.5 μg of P. gingivitis TL resulted in enhanced severity of EAE, as illustrated in FIG. 7. Component HPLC fractions of the TL were examined individually. The examination showed that the fraction containing greater than 95% PE DHC most consistently enhanced EAE. Administering 2.5 μg, 250 ng, and even 25 ng of PE DHC led to enhanced disease, with 250 ng being the most efficient. A single 250 ng i.p. injection of the PE DHC fraction consistently enhanced the severity of EAE and often led to earlier onset of disease. FIG. 7 illustrates one representative experiment of six similar studies, in which 250 ng of PE DHC was administered to WT mice. The cumulative results from these six experiments demonstrated that PE DHC-treated mice showed essentially a doubling in cumulative disease index (CDI) and mean daily disease compared with EtOH-treated mice, as illustrated by Table 1. In addition, WT PE DHC-treated mice showed a significantly earlier onset of disease when compared to WT EtOH-treated mice (p=0.008). While not reaching statistical significance, PE DHC-treated mice also showed an increase in incidence of disease, as illustrated in Table 1. Mean maximum severity did not differ significantly between the groups. Of note, we also administered the lipids to naïve mice that were not treated with the EAE-inducing protocol and observed these mice for signs of illness. Such mice never demonstrated EAE.

### Example 10

### PE DHC Enhances EAE in IL15−/− and IL-15Ra−/− Mice

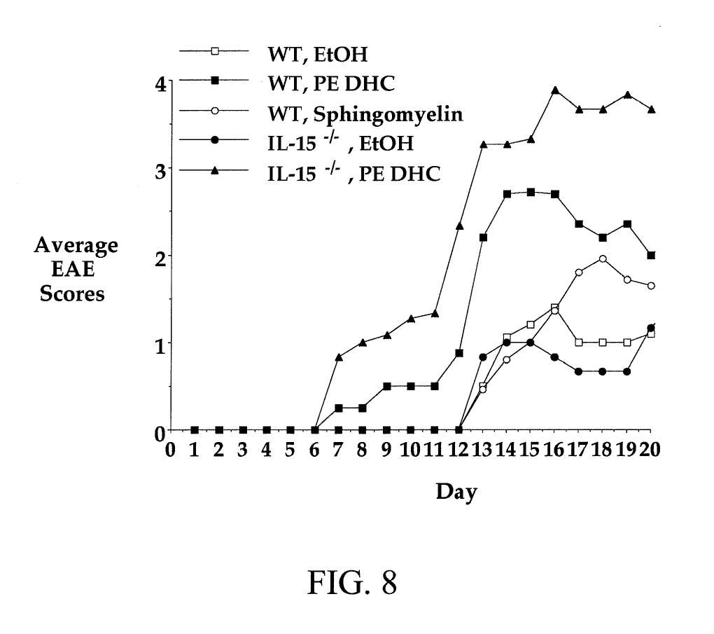

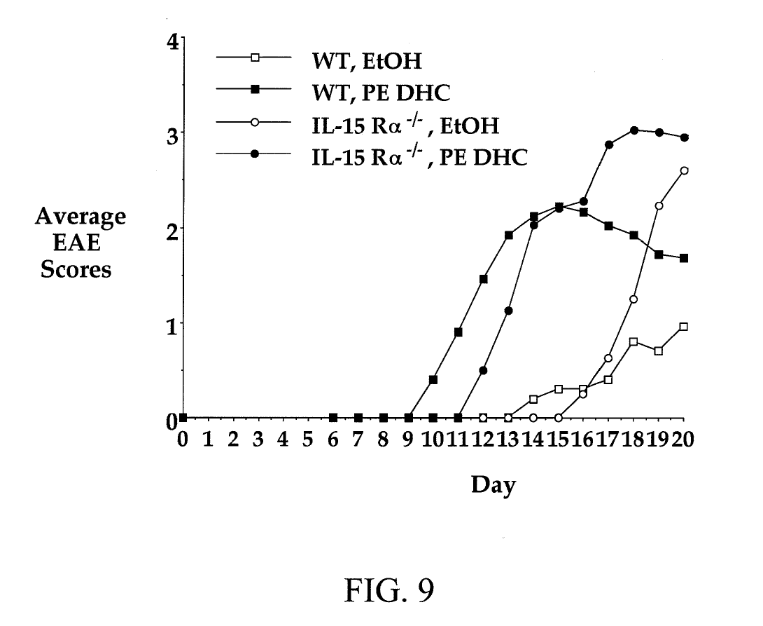

Mice deficient in either IL-15 (IL-15^{-/-} mice) or the IL-15 receptor α (IL-15Rα^{-/-} mice) are known to express very few identifiable NKT cells (Kennedy et al. “Reversible defects in natural killer and memory CD8 T cell lineages in interleukin 15-deficient mice,” J. Exp. Med. 191:771-780 (2000); Lodolce et al. “IL-15 receptor maintains lymphoid homeostasis by supporting lymphocyte homing and proliferation” Immunity 9:669-676 (1998). WT mice and either IL-15^{-/-} or IL-15Rα^{-/-} mice (both on a C57BL/6 background) were immunized for EAE and given a single i.p. injection of EtOH or PE DHC on Day 0. As in WT mice, PE DHC significantly enhanced EAE in both IL-15^{-/-} and IL-15Rα^{-/-} mice, inducing greater than a doubling of the CDI and mean daily disease compared with EtOH-treated IL-15^{-/-} and IL-15Rα^{-/-} mice (as shown Table 1). Additionally, IL-15^{-/-} and IL-15Ra^{-/-} PE DHC-treated mice showed an earlier onset of disease and increased incidence of disease compared to EtOH-treated mice, though only incidence of disease in IL-15Rα^{-/-} PE DHC-treated versus IL-15Rα^{-/-} EtOH-treated mice reached statistical significance (p=0.0285; as shown in Table 1). As with WT mice, mean maximum severity did not differ between the groups. FIGS. 8 and 9 show representative experiments using IL-15^{-/-} and IL-15Ra^{-/-} mice. The finding that PE DHC enhances EAE in IL-15^{-/-} and IL-15Rα^{-/-} mice indicates that PE DHC does not require NKT cells, the most common immune cells known to respond to sphingolipids, in order to mediate its disease-enhancing effect.

### Example 11

### PE DHC Enhancement of EAE is TLR2-Dependent

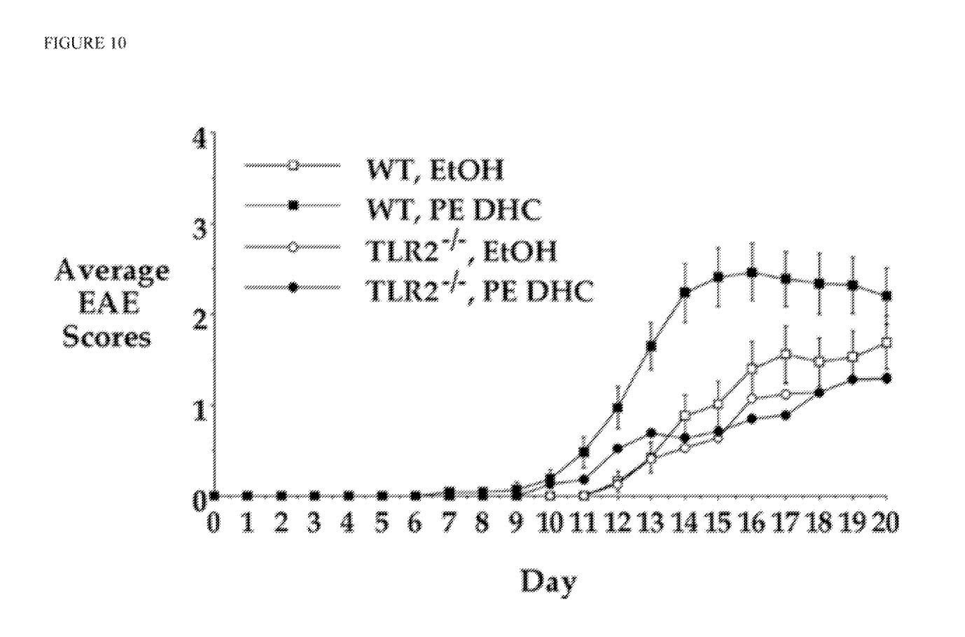

TLR-2 deficient (TLR2^{-/-}) mice were immunized with the standard EAE-inducing MOG protocol and administered a single i.p. injection of either EtOH or PE DHC on Day 0. In contrast to its effect on WT, IL-15^{-/-}, and IL-15Rα^{-/-} mice, PE DHC did not mediate enhancement of CDI or mean daily disease in TLR2^{-/-} mice. As seen in FIG. 10 (a composite of four experiments; n=15 mice) and in Table 1, PE DHC-treated TLR2^{-/-} mice demonstrated no statistically significant enhancement of EAE CDI, mean daily disease, disease incidence, mean maximal severity or day of onset when compared to EtOH-treated TLR2^{-/-} mice. These results indicated that TLR2 was required for PE DHC to mediate enhancement of EAE.

### Example 12

### PE DHC Enhancement of EAE was not a Result of Contamination with LPS or Lipid A

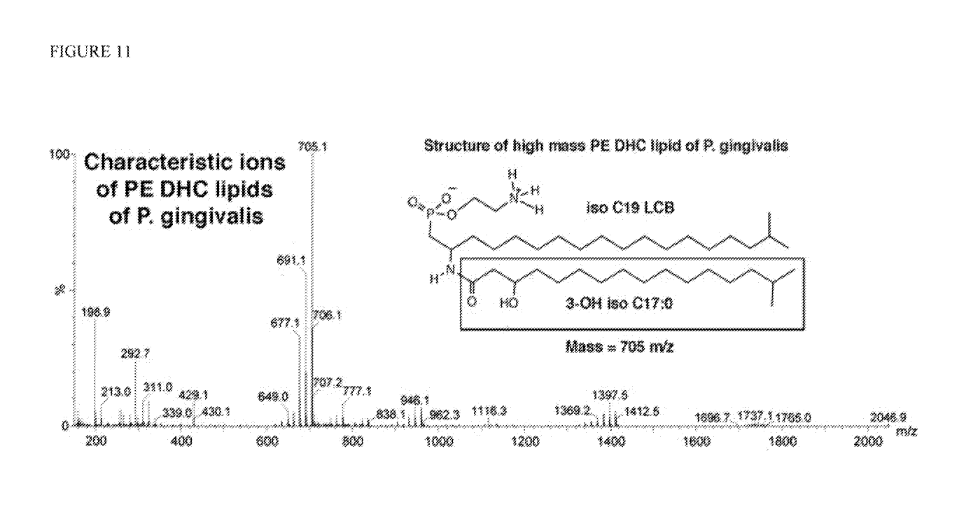

Since LPS preparations have been shown to influence the development of EAE, it was desirable to establish that PE DHC was not contaminated with Lipid A or LPS. The Bligh and Dyer phospholipid extraction procedure that was used for recovering the P. gingivalis lipids has previously been shown to exclude LPS of P. gingivalis from the organic solvent phase containing the total bacterial lipids. See Nichols “Distribution of 3-hydroxy iC17:0 in subgingival plaque and gingival tissue samples: Relationship to adult periodontitis” Infect. Immun. 62:3753-3760 (1994); Safavi & Nichols “Effect of calcium hydroxide on bacterial lipopolysaccharide” J. Endod. 19:76-78 (1993). Furthermore, P. gingivalis total lipids extracted by this method also did not contain Lipid A species known to be produced by P. gingivalis. The PE DHC lipid fraction was previously characterized using collisional electrospray-MS/MS studies, as described in Nichols et al. “Structures and biological activities of novel phosphatidylethanolamine lipids of Porphyromonas gingivalis,” J. Lipid Res. 47:844-853 (2006). Structural NMR studies were also used. Both studies confirmed the structural characteristics of the lipids and lack of both carbohydrate and protein contaminants in the relevant lipid fraction. In addition, contamination of the fraction with neutral LPS was unlikely because the HPLC separations used a polar column, and the relevant lipid was highly polar and therefore late eluting. All neutral lipid components eluted close to the void volume and were not recovered in the lipid fractions used in these studies. Electrospray-MS evaluation of all the major lipid classes purified by HPLC confirmed that these lipid fractions were not contaminated with Lipid A species of P. gingivalis LPS. Electrospray-MS of the PE DHC lipid fraction of P. gingivalis demonstrated that the characteristic dominant Lipid A negative ions (1195, 1435, 1449, 1690 and 1770 m/z) previously described for P. gingivalis. See Darveau et al. “Porphyromonas gingivalis lipopolysaccharide contains multiple lipid A species that functionally interact with both toll-like receptors 2 and 4” Infect. Immun. 72:5041-5051 (2004) and Reife et al. “Porphyromonas gingivalis lipopolysaccharide lipid A heterogeneity: differential activities of tetra- and penta-acylated lipid A structures on E-selectin expression and TLR4 recognition” Cell Microbiol. 8:857-868 (2006), were not recovered in this isolate, as illustrated in FIG. 11. Thus, the approach used for preparation of the lipids and the analyses of the lipid fractions ruled out the possibility that the P. gingivalis PE DHC fraction was contaminated with Lipid A or LPS.

### Example 13

### Administration of PE DHC Resulted in Increased Recovery of Bacterial Lipids in the Brains of Mice with EAE

The level of 3-OH isobranched (iso)C17:0 fatty acid was determined in brain specimens of mice with EAE treated with PBS, EtOH or PE DHC. The approach of measuring 3-OH isoC_{17:0} fatty acid in tissues was based on the concept that mammalian tissues, unlike bacteria, have no established biochemical pathway for de-novo synthesis of 3-OH isoC_{17:0} fatty acid. Thus, the recovery of 3-OH isoC_{17:0} fatty acid reflects the presence of bacterially-derived products in the tissue. 3-OH isoC_{17:0} is a constituent fatty acid of all phosphorylated dihydroceramide lipids of P. gingivalis. Nichols et al. (2004)

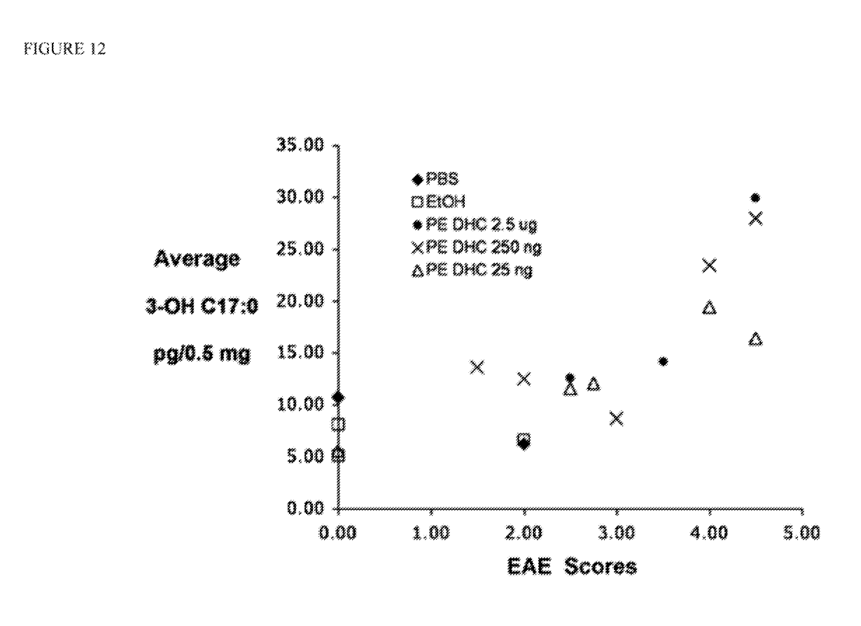

Mice treated with PBS, EtOH or with 25 ng, 250 ng, or 2.5 ug PE DHC were sacrificed after day 20 post-EAE immunization. The brains were removed, extracted for phospholipids, and fatty acid recovery was quantified by selected ion monitoring for fatty acid negative ions. FIG. 12 illustrates the average 3-OH isoC_{17:0} recovery (3 determinations/mouse brain sample) as a function of both the final grade of EAE and the treatment received by each mouse. The data were expressed as picograms of 3-OH isoC_{17:0} per 0.5 mg of total brain lipid extracted. The average S.E.M. for all determinations was +/−2.2 pg/0.5 mg total lipid. As illustrated in FIG. 12, lipids derived from the brains of control (PBS or EtOH-injected) mice showed low levels of recoverable 3-OH isoC_{17:0} fatty acid. These experimental data reflected cumulative exposure of normal mice to complex lipids and/or LPS derived from other commensal bacteria. The experimental results showed higher levels of 3-OH isoC_{17:0} fatty acid in mice that had received PE DHC and had a disease score greater than 3.0, as illustrated in FIG. 12. Linear regression analysis revealed that the correlation between EAE disease score and brain 3-OH isoC_{17:0} fatty acid was directly associated with the dose of PE DHC injected: the strongest correlation (regression coefficient or slope) was seen with the highest dose of PE DHC (2.5 ug, y=11.578+8.690x, R²=0.818), the next strongest with the middle dosage (250 ng, y=2.168+5.014x, R²=0.620), and the weakest correlation with the lowest dose of PE DHC (25 ng, y=3.789+3.062x, R²=0.808).

### Example 14

### PE DHC Activated APCs and Induced IL-6 Secretion In Vitro in a TLR2-Dependent Manner

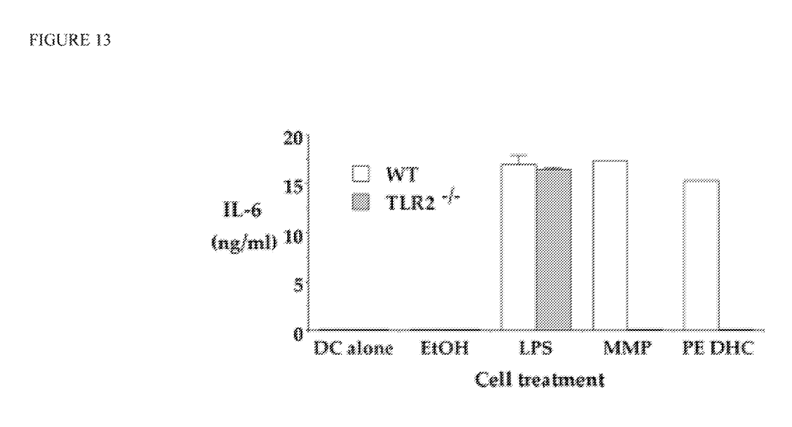

The effects of PE DHC on antigen presenting cell (APC) activation in vitro were examined. Dendritic cells (BMDCs) (>85% CD11c+) were derived from the bone marrow of WT or TLR2^{-/-} mice and cultured either alone or with EtOH, LPS, MMP (a TLR2 ligand), or PE DHC. After 18 Ins supernatants were assayed for IL-6. As illustrated in FIG. 13, stimulating WT BMDCs in the presence of LPS or MMP resulted in IL-6 secretion. Culturing TLR2^{-/-} BMDCs in the presence of LPS also resulted in IL-6 secretion, but culturing in the presence of MMP did not. WT BMDCs in the presence of PE DHC demonstrated levels of IL-6 secretion that were almost equivalent to that seen with LPS. However, in contrast to its effects on WT BMDCs, culturing PE DHC with TLR2^{-/-} BMDCs did not result in IL-6 secretion. BMDCs were also assayed for expression of the surface activation markers B7.2 and MHC class II. It was found that PE DHC increased MHC II and B7.2 expression on WT but not TLR2^{-/-} BMDCs. These results indicated that PE DHC can activate DCs and in a TLR2-dependent manner.

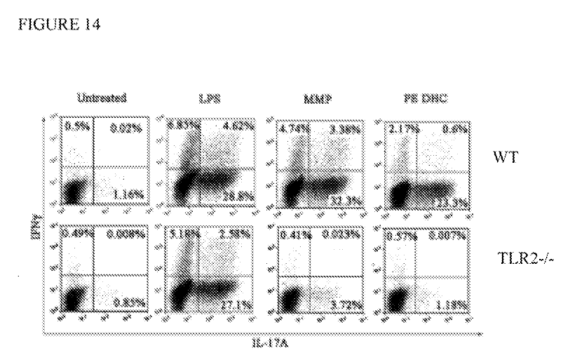

PE DHC's ability to induce IL-6 secretion was characterized by testing its ability to induce Th17 T cell generation from cultures of naïve CD4+CD25-T cells activated in the presence of APCs (T cell depleted splenocytes; Tds) and TGF-β. See Bettelli et al. “T(H)-17 cells in the circle of immunity and autoimmunity” Nat. Immunol. 2007, 8:345-350. Adding PE DHC resulted in the generation of Th17 T cells in cultures containing WT but not TLR2^{-/-} Tds, as illustrated in FIG. 14. These results further confirmed that PE DHC can induce IL-6 secretion from APCs in a TLR2-dependent manner. When taken together, these results indicated that PE DHC mediates its in vitro and in vivo effects through TLR2-dependent mechanisms.

### Example 15

### PE DHC Decreased the Percentage of CD4+Foxp3+Spinal Cord Tregs

To characterize mechanisms by which PE DHC may enhance autoimmune disease in vivo, the experimental studies tested whether the PE DHC-mediated enhancement of EAE was associated with alterations in T cell populations at a site of disease. Mice were immunized with the usual EAE-inducing protocol and treated on Day 0 with EtOH or PE DHC (250 ng i.p.). Within 5 days after onset of EAE, mice were sacrificed and exsanguinated, their spinal cords were removed, and the mononuclear cells were derived from the spinal cords. These cells were analyzed directly for CD4 and Foxp3 expression by flow cytometry or were stimulated with PMA and ionomycin for 4 hours and then, gating on Thy1.2+ cells, analyzed for intra-cellular interferon gamma (IFNγ) and IL-17 by flow cytometry. After sampling WT mice from three separate experiments, no significant difference were found in the total number of mononuclear cells obtained from the spinal cords of EtOH versus PE DHC-treated mice. In addition, the percentages of spinal cord-derived CD4+ T cells staining for either intra-cellular IFNγ or IL-17 (or cells expressing both cytokines) were not significantly different between EtOH and PE DHC-treated mice. However, the percentage of CD4+ T cells within the total mononuclear cell populations derived from the spinal cords of PE DHC-treated mice was, on average, greater than the percentage in EtOH-treated mice (as illustrated in Table 2). Moreover, while this increase in percentage of CD4+ T cells from PE DHC spinal cords did not reach statistical significance, a statistically significant decrease was observed in the mean percentage of spinal cord CD4+ T cells that were Foxp3+ (theoretically representing regulatory T cells; [Tregs]) in the PE DHC-treated mice (p=0.0397) (as illustrated Table 2). The mean percentage of spinal cord cells that were CD4+ was 41% in EtOH-treated mice and, on average, 6.7% of these were Foxp3+. In contrast, the mean percentage of spinal cord cells that were CD4+ T cells was 52% in PE DHC-treated mice and, on average, 4.3% of these were Foxp3+. It has been reported in Korn et al. “Myelin-specific regulatory T cells accumulate in the CNS but fail to control autoimmune inflammation” Nat. Med. 13:423-431 (2007) that the percentage of spinal cord CD4+ Foxp3+ T cells increased as the disease progressed. On average, the PE DHC-treated mice from which spinal cord cells were derived had a slightly longer duration of disease than did the EtOH-treated mice (1.5 days longer; as illustrated Table 2). Based on this observation, it was unlikely that the decrease in the percentage of Foxp3+ in PE-DHC-treated mice was related to differences in disease duration.

Different arrangements and combinations of the elements and the features described herein are possible. Similarly, some features and subcombinations are useful and may be employed without reference to other features and subcombinations. Embodiments of the invention and examples have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

TABLE 1
EAE disease assessment
A. The cumulative disease index (CDI) was obtained by summing the
daily average disease scores of each experimental group through Day 20.
A mean of these daily disease scores (Mean Daily Disease—MDD) was
calculated based on the 20 days of observation. The MDD scores
were compared using the Wilcoxin Signed Rank tests for two samples.
n is the total number of mice studied in each experimental group.
B. Mean incidence of disease is represented as a percentage and was
calculated by dividing the number of mice within each group that
developed clinical signs of EAE by the total number of mice in that
group. Disease incidence frequencies were compared using Chi
square analysis. Mean maximum severity of EAE was calculated for
mice that developed EAE by taking the highest score observed for
each mouse in the 20-day observation period and averaging these
values among mice in the same group. Statistical significance was
determined using the Wilcoxin Signed Rank test. Mean day of onset of
EAE was calculated for mice that developed EAE by using the first day
of observance of signs of EAE as the value and averaging these
values among mice in the same group. Statistical significance was
determined using the Student's t-test.
A.

| | EtOH | | PE DHC | | | |
|---|---|---|---|---|---|---|
| Mouse Strain | CDI | MDD | CDI | MDD | P value | n |
| Wild Type | 10.1 | 0.5 | 19.8 | 1.0 | 0.001 | 28 |
| TLR2^{-/-} | 7.6 | 0.4 | 8.3 | 0.4 | 0.306 | 15 |
| IL-15^{-/-} | 9.4 | 0.5 | 24.5 | 1.2 | 0.001 | 10 |
| IL-15Rα^{-/-} | 7.0 | 0.4 | 18.7 | 0.9 | 0.0077 | 12 |

B.

| | EtOH | PE DHC | EtOH | PE DHC | EtOH | PE DHC |
|---|---|---|---|---|---|---|
| | Mean | | Mean Maximum | | Mean Day | |
| Mouse Strain | Incidence | | Severity | | of Onset | |
| Wild Type | 58.6 | 75 | 3.2 | 3.6 | 14.4 | 12.1 |
| TLR2^{-/-} | 42.1 | 46.6 | 3.0 | 2.9 | 14.4 | 14.7 |
| IL-15^{-/-} | 60 | 90 | 3.1 | 3.7 | 14.7 | 13.0 |
| IL-15Rα^{-/-} | 66 | 100 | 2.7 | 2.9 | 15.1 | 13.8 |

TABLE 2
Spinal Cord T cells
Mice were sampled from 3 different experiments and sacrificed 1-5 days
after onset of signs of EAE. Mononuclear cells were derived from the
spinal cords, stained for CD4 and Foxp3, and evaluated by flow
cytometry. % CD4 represents the % CD4+ T cells within the total spinal
cord mononuclear cells. % Foxp3 represents the The PE DHC fraction
altered the composition of cells infiltrating the spinal cords of mice
with EAE. % Foxp3+ T cells after gating on CD4+ T cells.

| | DAYS AFTER | | | |
|---|---|---|---|---|
| Treatment- | ONSET | DISEASE | % CD4+ in | % Foxp3+ in |
| mouse | of EAE | GRADE | spinal cord | spinal cord |
| ETOH-1 | 2 | 1.0 | 49.60 | 8.31 |
| ETOH-2 | 4 | 2.8 | 55.23 | 5.71 |
| ETOH-3 | 5 | 3.3 | 49.40 | 5.57 |
| ETOH-4 | 2 | 2.7 | 44.88 | 7.52 |
| ETOH-5 | 1 | 1.0 | 32.98 | 6.01 |
| ETOH-6 | 1 | 2.0 | 15.18 | 7.06 |
| | | | Mean = 41.21 | Mean = 6.70 |
| | | | +/−14.78 | +/−1.11 |
| PE DHC-1 | 5 | 3.3 | 72.00 | 4.53 |
| PE DHC-2 | 4 | 3.5 | 38.00 | 7.52 |
| PE DHC-3 | 5 | 2.9 | 68.86 | 2.75 |
| PE DHC-4 | 4 | 3.3 | 49.54 | 3.45 |
| PE DHC-5 | 2 | 2.9 | 40.83 | 3.21 |
| PE DHC-6 | 4 | 3.0 | 46.50 | 4.27 |
| | | | Mean = 52.62 | Mean = 4.29 |
| | | | +/−14.42 | +/−1.72 |
| | | | p = 0.1635 | p = 0.0397 |

1. A method for detecting an inflammatory or an autoimmune condition, comprising:
analyzing occurrence of bacterial lipids in a lipid-containing sample obtained from a human or an animal;
comparing the occurrence of the bacterial lipids in the sample with occurrence of the bacterial lipids in a comparable reference sample; and,
detecting the inflammatory or the autoimmune condition based on the difference in occurrence of the bacterial lipids between the sample and the reference sample.

2-3. (canceled)

4. The method of claim 1, wherein the step of analyzing comprises identifying the bacterial lipids.

5. The method of claim 1, wherein the step of analyzing comprises quantitating the bacterial lipids.

6. The method of claim 1, wherein the step of analyzing comprises determining one or more quantitative relationships among categories of the bacterial lipids in the sample.

7-17. (canceled)

18. The method of claim 1, wherein the step of analyzing comprises performing mass-spectrometry analysis of the sample.

19. (canceled)

20. The method of claim 1, wherein the step of analyzing comprises contacting the sample with an antibody specific to a bacterial lipid under conditions allowing for specific binding of the antibody to the bacterial lipid.

21-23. (canceled)

23. The method of claim 1, wherein the bacterial lipids comprise phosphorylated dihydroceramides (PDHCs).

24. The method of claim 23, wherein the PDHCs comprise phosphoethanolamine dihydroceramides (PE DHCs) and phosphoglycerol dihydroceramides (PG DHCs).

25. (canceled)

26. The method of claim 1 wherein the autoimmune or the inflammatory condition is multiple sclerosis (MS).

27. The method of claim 26, wherein the sample is obtained from a human, and MS is detected based on an increased ratio of phosphoethanolamine dihydroceramides (PE DHCs) and phosphoglycerol dihydroceramides (PG DHCs) in the sample, as compared to the comparable sample obtained from a non-MS human subject.

28-31. (canceled)

32. A lipid-specific antibody capable of specific binding to a PDHC lipid category.

33-35. (canceled)

36. A method of using the antibody of claim 32 to modulate an immune response in a human or an animal.

37-41. (canceled)

42. A composition for modulating an immune response in an animal or a human, comprising a bacteria-originated lipid.

43. (canceled)

44. The composition of claim 42, wherein the bacteria-originated lipid is PDHC.

45. A method of modulating an immune response in an animal, a human, or in an animal or human cell or tissue, comprising administering to the animal, the human, or to the animal or human cell or tissue a composition of claim 42.

46-52. (canceled)

53. A method of modulating a toll-like receptor dependent pathway in an animal or a human, or in an animal or human cell or tissue, comprising administering to the animal, the human, or to the animal or human cell or tissue a composition of claim 42.

54-80. (canceled)

81. A method for detecting an inflammatory or an autoimmune condition in a human or an animal, comprising
identifying and quantifying bacterial lipids in a lipid-containing sample obtained from the human or the animal;
calculating one or more quantitative relationships between two or more categories of the bacterial lipids in the sample;
detecting the inflammatory or the autoimmune condition based on the one or more quantitative relationships.

82. The method of claim 81, wherein the two or more categories of the bacterial lipids comprise phosphoethanolamine dihydroceramides (PE DHCs) and phosphoglycerol dihydroceramides (PG DHCs).

83. The method of claim 82, wherein the inflammatory or the autoimmune condition is detected based on a ratio of PG DHC to PE DHC.

84. The method of claim 83, wherein the inflammatory or the autoimmune condition is multiple sclerosis.
